(19) United States
(12) Patent Application Publication
(10) Pub. No.: US 2015/0366813 A1
(43) Pub. Date: Dec. 24, 2015

(54) LIQUID-FILLED IMMEDIATE RELEASE SOFT GELATIN CAPSULES

(71) Applicant: Banner Life Sciences LLC, High Point, NC (US)

(72) Inventors: Rishi Thakur, Ellicott City, MD (US); George Vamvakas, Greensboro, NC (US); Tatyana Dyakonov, Greensboro, NC (US); Aqeel A Fatmi, High Point, NC (US)

(21) Appl. No.: 14/744,059

(22) Filed: Jun. 19, 2015

Related U.S. Application Data
(60) Provisional application No. 62/014,714, filed on Jun. 20, 2014.

(51) Int. Cl. A61K 9/48; A61K 31/4439

(52) U.S. Cl. CPC A61K 9/4808; A61K 31/4439; A61K 9/4825; A61K 9/4875; A61K 9/4858

(57) ABSTRACT

Described herein are oral pharmaceutical compositions comprising labile, air-, moisture-, or heat-sensitive active pharmaceutical ingredients and methods for making the same. In particular, immediate release capsules comprising non-aqueous fills comprising moisture-sensitive active pharmaceutical ingredients are described.

### CROSS REFERENCE TO RELATED APPLICATIONS

This application claims priority to U.S. Provisional Patent Application No. 62/014,714, filed Jun. 20, 2014, which is incorporated by reference herein in its entirety. This application is related to International Patent Application No. PCT/US2015/______, filed on Jun. 19, 2015, which is incorporated by reference herein in its entirety.

### TECHNICAL FIELD

Described herein are oral pharmaceutical compositions comprising labile, air-, moisture-, or heat-sensitive active pharmaceutical ingredients and methods for making the same. In particular, immediate release capsules comprising non-aqueous fills and active pharmaceutical ingredients are described.

### BACKGROUND

Many active pharmaceutical ingredients (APIs) are labile or sensitive to light, oxidation, moisture, or heat. Further, some compounds are poorly soluble. These attributes can cause difficulties in the manufacturing process and reduce the shelf life of the resulting product.

Accordingly, there is a need for oral pharmaceutical compositions that protect the active pharmaceutical ingredient from light, air, moisture, and heat during manufacturing and storage. The absorption of moisture by the API during processing can reduce the content of the pharmaceutically active substance in the dosage form because of the increased weight caused by the uptake of water. Pharmaceutical compositions with a tendency to absorb moisture need to be protected from moisture during storage, e.g., by the addition of suitable drying agents or by storing the drug in an environment where it is protected from moisture. In addition, the uptake of moisture may reduce the content of pharmaceutically active substance during manufacture if the pharmaceutical substance is exposed to the environment without being protected from moisture in any way. Preferably, therefore, a pharmaceutically active substance should be only slightly hygroscopic.

Pharmaceutical substances that are sensitive to moisture and light often undergo hydrolytic degradation or photodegradation, respectively. Accordingly, moisture and light sensitive substances often undergo polymorphic form transformations (e.g., enantiomeric conversion). These processes can result in the generation of degradation products and impurities that reduce the efficacy, potency, and activity of the pharmaceutical substance requiring additional dosages of the pharmaceutical substance. Taking additional dosages of pharmaceuticals places an undue burden on patients. In addition, active pharmaceutical degradation products can act antagonistically to the drug substance or even become genotoxic, resulting in unforeseen side effects in patient populations.

Currently, there are limited options for protecting active drug substances from moisture degradation. For example, only slightly hygroscopic or non-hygroscopic excipients are chosen to help protect the active drug substances during formulation, which unduly limits the range of pharmaceutical ingredients for further formulating pharmaceutical compositions having moisture sensitive drugs. Other methods utilize special external polymeric coatings (e.g., Kollicoat), which function to reduce the absorption of external water by soft gel capsules. However, external coatings do not prevent migration of water from the capsule shell to the active drug substance and are often complicated to apply and result in an increased variation in coating. These variations in coatings result in decreased drug stability between soft gel capsules, which affects proper dosing.

In addition, moisture sensitive active drug substances are often incorporated into tablets or encapsulated within hard capsules to eliminate the migration of moisture. However, there is a demand from consumers for pharmaceutical compositions having soft gel capsules, which are easier to ingest than hard capsules or tablets. Thus, new pharmaceutical soft gel formulations which address the physicochemical requirements and that are cost effective and feasibly manufactured are needed. Furthermore, the problem addressed by the oral pharmaceutical composition described herein is to provide a pharmaceutically active substance comprising soft gel capsules which not only is characterized by a high pharmacological potency, but also satisfies the abovementioned physicochemical requirements as far as possible.

### SUMMARY

One embodiment described herein is an oral pharmaceutical composition comprising a soft capsule comprising an immediate release matrix comprising: (a) one or more lipid or lipophilic vehicles; (b) one or more active pharmaceutical ingredients; (c) one or more pharmaceutically acceptable excipients; and wherein the one or more active pharmaceutical ingredients are suspended within the matrix and the matrix inhibits moisture from contacting the active pharmaceutical ingredient. In one aspect described herein, the one or more active pharmaceutical ingredients comprise moisture-sensitive active ingredients, light-sensitive active ingredients, or a combination thereof. In another aspect described herein, the one or more active pharmaceutical ingredients comprise a moisture-sensitive active ingredient. In another aspect described herein, the moisture-sensitive active ingredient comprises dabigatran, aspirin, or diclofenac. In another aspect described herein, the active pharmaceutical ingredient comprises dabigatran. In another aspect described herein, the one or more lipid or lipophilic vehicles comprise about 35% to about 90% of the total mass of the matrix. In another aspect described herein, the one or more lipid or lipophilic vehicles comprise soybean oil, fish oil, polyunsaturated fatty acids, Miglyol® 812, Paceol™, or Capryol™, or a combination thereof. In another aspect described herein, the one or more active pharmaceutical ingredients comprise about 10% to about 40% of the total mass of the matrix. In another aspect described herein, the one or more pharmaceutically acceptable excipients comprise about 0.01% to about 15% of the total mass of the matrix. In another aspect described herein, the one or more pharmaceutically acceptable excipients comprises one or more non-ionic surfactants; one or more hydrophilic ionic polymers, one or more hygroscopic polymers, one or more pH buffering agents, one or more neutralizing agents, one or more antioxidants, one or more water-binding excipients, or one or more absorption enhancers, or a combination thereof. In another aspect described herein, the one or more pharmaceutically acceptable excipients comprises: antioxidants, non-ionic surfactants, absorption enhancers, or combinations thereof. In another aspect described herein, the non-ionic surfactants comprise about 5% to about 15% of the total mass of the matrix. In another aspect described herein, the non-ionic surfactants comprise about 35% to about 80% of the total mass of the matrix. In another aspect described herein, the non-ionic surfactants comprise Cremophor® RH 40, TWEEN® 80, Span® 80, Labrasol®, Lauroglycol™, or lecithin, or a combination thereof. In another aspect described herein, the one or more antioxidants comprises about 0.01% to about 2% of the total mass of the matrix. In another aspect described herein, the one or more antioxidants comprise tocopherol, BHT, BHA, citric acid, ascorbic acid, or phenolic diterpene, or a combination thereof. In another aspect described herein, the one or more absorption enhancers comprise Vitamin E TPGS. In another aspect described herein, the one or more absorption enhancers comprise about 1% to about 7% of the total mass of the matrix. In another aspect described herein, the matrix comprises soybean oil, BHT, and dabigatran. In another aspect described herein, soft capsule shell comprises one or more film forming polymers, one or more plasticizers, and one or more solvents. In another aspect described herein, the soft capsule shell further comprises optionally, an opacifying agent, a coloring agent, or a pharmaceutical excipient. In another aspect described herein, the soft capsule shell comprises: (a) about 43% of one or more film-forming polymers; (b) about 20% of one or more plasticizers; (c) about 37% of one or more solvents; In another aspect described herein, the soft capsule shell further comprises: (d) optionally, about 0.7% of one or more opacifying agents; and (e) optionally, about 0.1% of one or more coloring agents. In another aspect described herein, the matrix comprises: (a) about 80% to about 90% soybean oil; (c) about 9% to about 20% dabigatran; and (b) about 0.01% BHT.

Another embodiment described herein is an oral pharmaceutical composition comprising a soft capsule shell comprising an immediate release matrix, the matrix comprising: (a) about 81% soybean oil; (b) about 0.01% BHT; and (c) about 19% dabigatran; wherein the dabigatran is suspended with in the matrix and the matrix retards moisture from contacting dabigatran; and the soft capsule shell comprises one or more film forming polymers, one or more plasticizers, and one or more solvents. In one aspect described herein, the soft capsule shell comprises: (a) about 43% of one or more film-forming polymers; (b) about 20% of one or more plasticizers; and (c) about 37% of one or more solvents. In another aspect described herein, the soft capsule shell comprises gelatin, glycerol, and water.

Another embodiment described herein is an oral pharmaceutical composition comprising a soft capsule shell comprising an immediate release matrix comprising: (a) about 90% soybean oil; (b) about 0.01% BHT; and (c) about 9% dabigatran; wherein the dabigatran is suspended with in the matrix and the matrix retards moisture from contacting dabigatran; and the soft capsule shell comprises one or more film forming polymers, one or more plasticizers, and one or more solvents. In one aspect described herein, the soft capsule shell comprises: (a) about 43% of one or more film-forming polymers; (b) about 20% of one or more plasticizers; and (c) about 37% of one or more solvents. In another aspect described herein, the soft capsule shell comprises gelatin, glycerol, and water.

Another embodiment described herein is a method for manufacturing an oral immediate release soft capsule shell and matrix fill comprising: (a) providing a matrix comprising: (i) about 80% to about 90% soybean oil; (ii) about 9% to about 20% dabigatran; and (ii) about 0.01% BHT; (b) providing a soft capsule gel mass comprising: (i) about 43% of at least one film-forming polymer; (ii) about 20% of at least one plasticizer; and (iii) about 37% of a solvent; (c) casting the soft capsule gel mass into films using heat-controlled drums or surfaces; and (d) forming a soft capsule comprising the matrix composition using rotary die encapsulation technology.

Another embodiment described herein is a soft capsule dosage form comprising an immediate release matrix produced by the any of the methods described herein. In one aspect described herein, the dosage form dissolves in simulated gastric fluid (pH 1.2) within about 10 minutes.

Another embodiment described herein is an oral pharmaceutical composition comprising a soft capsule shell comprising an immediate release matrix, the matrix comprising: (a) about 81% soybean oil; (b) about 0.01% BHT; and (c) about 19% dabigatran; and the soft capsule shell comprising: (d) about 43% gelatin; (e) about 20% glycerol; and (f) about 37% water; wherein the dabigatran is suspended with in the matrix and the matrix retards moisture from contacting dabigatran.

Another embodiment described herein is an oral pharmaceutical composition comprising a soft capsule shell comprising an immediate release matrix, the matrix comprising: (a) about 90% soybean oil; (b) about 0.01% BHT; and (c) about 9% dabigatran; and the soft capsule shell comprising: (a) about 43% gelatin; (b) about 20% glycerol; and (c) about 37% water; wherein the dabigatran is suspended with in the matrix and the matrix retards moisture from contacting dabigatran.

Another embodiment described herein is a method for treating, ameliorating the symptoms of, or delaying the onset of a medical condition by providing a subject in need thereof with an oral pharmaceutical composition comprising: (a) one or more lipid or lipophilic vehicles; (b) one or more light or water sensitive active pharmaceutical ingredients; and (c) one or more pharmaceutically acceptable excipients; wherein the one or more active pharmaceutical ingredients are suspended within the matrix fill and the matrix inhibits moisture from contacting the active pharmaceutical ingredients. In another aspect described herein, the medical condition comprises a coagulation disorder comprising: stroke, ischemic stroke, hemorrhagic stroke, pulmonary embolism, clotting in patients with non-valvular atrial fibrillation, stroke in patients with non-valvular atrial fibrillation, systemic embolism in patients with non-valvular atrial fibrillation, or deep vein thrombosis or a combination thereof. In one aspect described herein, the subject in need thereof has been previously treated with a parenteral anticoagulant. In another aspect described herein, the parenteral anticoagulant comprises heparin, fondaparinux, danaparoid, recombinant hirudins, bivalirudin, or argatroban.

Another embodiment described herein is a kit for dispensing an oral pharmaceutical composition comprising: (a) at least one soft capsule comprising an immediate release matrix comprising: (i) one or more lipid or lipophilic vehicles; (ii) one or more light or water sensitive active pharmaceutical ingredients; and (iii) one or more pharmaceutically acceptable excipients; (b) at least one receptacle comprising a tamper evident, moisture proof packaging that reduces the ability of removing the oral pharmaceutical composition comprising blister or strip packs, aluminum blister, transparent or opaque polymer blister with pouch, polypropylene tubes, colored blister materials, tubes, bottles, and bottles optionally containing a child-resistant feature, optionally comprising a desiccant, such as a molecular sieve or silica gel; (c) optionally, an insert comprising instructions or prescribing information for the active pharmaceutical ingredient.

Another embodiment described herein is a composition for treating, ameliorating the symptoms of, or delaying the onset of a medical condition comprising: (a) one or more lipid or lipophilic vehicles; (b) one or more light or water sensitive active pharmaceutical ingredients; and (c) one or more pharmaceutically acceptable excipients; wherein the one or more active pharmaceutical ingredients are suspended within the matrix fill and the matrix inhibits moisture from contacting the active pharmaceutical ingredients. In one aspect described herein, the medical condition comprises a coagulation disorder comprising: stroke, ischemic stroke, hemorrhagic stroke, pulmonary embolism, clotting in patients with non-valvular atrial fibrillation, stroke in patients with non-valvular atrial fibrillation, systemic embolism in patients with non-valvular atrial fibrillation, or deep vein thrombosis or a combination thereof.

Another embodiment described herein is a means for inhibiting moisture from contacting one or more active pharmaceutical ingredients comprising a pharmaceutical composition comprising a soft capsule shell comprising an immediate release matrix, the matrix comprising: one or more lipid or lipophilic vehicles; one or more active pharmaceutical ingredients; and one or more pharmaceutically acceptable excipients. In one aspect described herein, the pharmaceutical composition comprises: a matrix comprising: (a) about 80% to about 90% soybean oil; (b) about 9 to about 20% dabigatran; and (c) about 0.01% BHT; a soft capsule gel mass comprising: (d) about 43% of at least one film-forming polymer; (e) about 20% of at least one plasticizer; and (f) about 37% of a solvent.

### BRIEF DESCRIPTION OF THE DRAWINGS

FIG. 1. Manufacturing scheme demonstrating the methods used to generate the matrix fills of the pharmaceutical compositions.

FIG. 2. Dissolution profiles of three application batches of DEM in a soft gel capsule.

FIG. 3. Effect of DEM etexilate particle size on DEM dissolution kinetics.

FIG. 4. Dissolution kinetics of DEM in a soft gel capsule in 0.01 N HCl.

FIG. 5. The effect of different surfactants on DEM release kinetics.

FIG. 6. Effect of CTAB on DEM dissolution kinetics.

FIG. 7. Effect of storage time on DEM dissolution kinetics.

FIG. 8. Dissolution kinetics of DEM soft capsule comprising the composition of Table 12.

### DETAILED DESCRIPTION

The term “active ingredient” or “active pharmaceutical ingredient” or “active pharmaceutical agent” as used herein refers to an agent, active ingredient, compound, or substance, compositions, or mixtures thereof, that provide a pharmacological, often beneficial, effect. Reference to a specific active ingredient includes, where appropriate, the active ingredient and any of its pharmaceutically acceptable salts or esters.

The terms “dosage” or “dose” denote any form of the active ingredient formulation that contains an amount sufficient to produce a therapeutic effect with a single administration. The dosage form used herein is for oral administration. The preferred oral dosage forms are soft capsules or enteric soft capsules.

As used herein, the term “pharmaceutical composition” refers to a composition comprising at least on active ingredient, nutraceutical, nutritional, or vitamin. In some embodiments described herein, a pharmaceutical composition comprises a soft capsule shell having been formed into a capsule, for example, using rotary die encapsulation comprising one or more polyunsaturated fatty acids, optionally with one or more vitamins, antioxidants, or other active ingredients.

The term “formulation” or “composition” as used herein refers to the active pharmaceutical ingredient or drug in combination with pharmaceutically acceptable excipients. This includes orally administrable formulations as well as formulations administrable by other means.

The term “room temperature” as used herein refers to common ambient temperatures found in pharmaceutical laboratories ranging from about 20° C. to about 27° C.

The term “controlled release” as used herein means an active pharmaceutical ingredient that is not immediately released. This term encompasses the terms “delayed release,” “modified release,” “sustained release,” and “extended release.”

The term “delayed release” as used herein refers to a composition that releases an active ingredient according to a desired profile over an extended period under physiological conditions or in an in vitro test. By “extended period” it is meant a continuous period of time of at least about 20 minutes, about 30 minutes, about 1 hour; about 2 hours; about 4 hours; about 6 hours; about 8 hours; about 10 hours; or about 12 hours.

The term “modified release” as used herein means a pharmaceutical formulation that releases the drug substance at a slower rate than does an immediate release formulation under physiological conditions or in an in vitro test.

The term “sustained” release” as used herein means release over an extended period of time, for example, minutes, hours, or days, such that less than all the active ingredient is released initially. A sustained release rate may provide, for example, a release of a certain specified amount of a drug or active ingredient from a dosage form, over a certain time period, under physiological conditions or in an in vitro test.

The term “extended release” as used herein refers to a composition that releases an active ingredient over an extended period, such as over at least about 20 minutes, about 30 minutes, about 1 hour; about 2 hours; about 4 hours; about 6 hours; about 8 hours; about 10 hours; about 12 hours; about 14 hours; about 16 hours; about 18 hours; about 20 hours about 24 hours; or even longer; specifically over a period of at least 18 hours under physiological conditions or in an in vitro assay.

The term “immediate release” as used herein refers to an immediate release of the active ingredient from the matrix fill over a shortened period of time, such as over 1 minute, 2 minutes, 3 minutes, 4 minutes, 5 minutes, 6 minutes, 7 minutes, 8 minutes, 9 minutes, 10 minutes, 15 minutes, 20 minutes, or 30 minutes.

As used herein, the term “fatty acid” refers to any carboxylic acid having a long aliphatic chain that can be either saturated or unsaturated. The term fatty acid further encompasses any fish oil described herein and any saturated, polyunsaturated, monounsaturated, or any omega-3, -6, -7, or -9 fatty acid.

The term “absolute bioavailability” as used herein refers to the fraction of a drug or active pharmaceutical ingredient absorbed through non-intravenous administration (e.g., oral administration) to intravenous administration of the same drug or active pharmaceutical ingredient.

As used herein, the term “polyunsaturated fatty acid” (“PUFA”) refers to a long chain fatty acid that contains more than one double bond in the backbone of the chain. The term encompasses esters, re-esterified triglycerides, or salts thereof.

As used herein, the term “monounsaturated fatty acid” refers to a long chain fatty acid that contains only one double bond in the backbone of the chain. The term encompasses esters, re-esterified triglycerides, or salts thereof.

The term “Cₘₐₓ” as used herein refers to the maximum observed blood (plasma, serum, or whole blood) concentration or the maximum blood concentration calculated or estimated from a concentration to time curve, and is expressed in units of mg/L or ng/mL, as applicable.

The term “Cₘᵢₙ” as used herein refers to the minimum observed blood (plasma, serum, or whole blood) concentration or the minimum blood concentration calculated or estimated from a concentration to time curve, and is expressed in units of mg/L or ng/mL, as applicable.

The term “C_{avg}” as used herein refers to the blood (plasma, serum, or whole blood) concentration of the drug within the dosing interval, is calculated as AUC/dosing interval, and is expressed in units of mg/L or ng/mL, as applicable.

The term “Tₘₐₓ” as used herein refers to the time after administration at which Cₘₐₓ occurs, and is expressed in units of hours (h) or minutes (min), as applicable.

The term “AUC_{0→τ}” as used herein refers to area under the blood (plasma, serum, or whole blood) concentration versus time curve from time zero to time tau (τ) over a dosing interval at steady state, where tau is the length of the dosing interval, and is expressed in units of h·mg/L or h·ng/mL, as applicable. For example, the term AUC_{0→12} as used herein refers to the area under the concentration versus time curve from 0 to 12 hours.

The term “AUC_{0→∝}” as used herein refers to the area under the blood (plasma, serum, or whole blood) concentration versus time curve from time 0 hours to infinity, and is expressed in units of h·mg/L or h·ng/mL, as applicable.

The term “AUCₒᵥₑᵣₐₗₗ” as used herein refers to the combined area under the blood (plasma, serum, or whole blood) concentration versus time curve, and is expressed in units of h·mg/L (or h·ng/mL) for at least one or more doses of the pharmaceutical compositions described herein. In one aspect, the “AUCₒᵥₑᵣₐₗₗ” refers to the combined area under the blood concentration versus time curve for at least two doses of the pharmaceutical compositions described herein.

The term “treating” refers to administering a therapy in an amount, manner, or mode effective to improve a condition, symptom, or parameter associated with a disorder.

The term “prophylaxis” refers to preventing or reducing the progression of a disorder, either to a statistically significant degree or to a degree detectable to one skilled in the art.

The term “substantially” as used herein means to a great or significant extent, but not completely.

The term “about” as used herein refers to any values, including both integers and fractional components that are within a variation of up to ±10% of the value modified by the term “about.” For example, the phrase “about 50%” is equivalent to any value ≈50±10%, e.g., 44.6%, 45%, 46%, 47%, 48%, 49%, 49.5%, 50%, 50.3%, 51%, 52%, 53%, 54%, 55%, inter alia.

As used herein, “a” or “an” means one or more unless otherwise specified.

Terms such as “include,” “including,” “contain,” “containing,” “has,” or “having,” and the like, mean “comprising.”

The term “or” can be conjunctive or disjunctive.

In one embodiment described herein, the pharmaceutical composition described herein comprises a soft capsule shell and a matrix fill comprising a lipid or lipophilic vehicle, one or more pharmaceutically acceptable excipients, and at least one or more active pharmaceutical ingredients. In one aspect, the pharmaceutically acceptable excipient comprises: one or more hydrophilic ionic polymers, one or more hygroscopic polymers, one or more pH buffering agents, one or more neutralizing agents, one or more antioxidants, one or more water-binding excipients, or one or more absorption enhancers.

In one embodiment described herein, the soft capsule matrix fill has the composition of Table 1, including all possible iterations of the specified ranges that provide 100% for the total weight percentage, including or excluding any optional colorings, flavorings, or excipients.

TABLE 1
Exemplary Immediate Release Matrix Composition

| | | Composition |
|---|---|---|
| Component | Exemplary Components | Range (%) |
| Lipid or lipophilic | Soybean oil, Fish oil, Miglyol ® | 40-90 |
| vehicle | Paceol ™, Capryol ™, | |
| | Capmul ® | |
| Water-binding excipient | Cyclodextrin, Aerosil ® 200, | 0.01-5 |
| | Syloid ® FP Silica | |
| Antioxidant | Tocopherols, BHT, BHA | 0.001-2 |
| Non-ionic surfactant | Lauroglycol ™, Labrasol ®, | 0.01-20 |
| | TWEEN ®, Span ® | |
| Active pharmaceutical | Dabigatran (DEM), | 10-40 |
| ingredient | Aspirin, Diclofenac | |
| Absorption enhancer | Vitamin E TPGS | 0.01-7 |

In another embodiment, the lipid or lipophilic vehicle comprises a liquid lipophilic vehicle, a semisolid lipophilic vehicle, or a mixture thereof. Suitable lipid or lipophilic vehicles include mineral oil; light mineral oil; natural oils (e.g., vegetable, corn, canola, sunflower, soybean, olive, coconut, cocoa, peanut, almond, cottonseed, persic, sesame, squalane, castor, cod liver, etc) hydrogenated vegetable oil; partially hydrogenated oils; beeswax; polyethoxylated beeswax; paraffin; normal waxes; medium chain medium chain monoglycerides, diglycerides and triglycerides; higher aliphatic alcohols; higher aliphatic acids; long chain fatty acids; saturated or unsaturated fatty acids; hydrogenated fatty acids; fatty acid glycerides; polyoxyethylated oleic glycerides; monoglycerides and diglycerides; mono-, bi- or tri-substituted glycerides; glycerol mono-oleate esters; glycerol mono-caprate; glyceryl monocaprylate; propylene glycol dicaprylate; propylene glycol monolaurate; glyceryl palmitostearate; glyceryl behenate; diethyleneglycol palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; fatty alcohols associated with polyethoxylate fatty alcohols; cetyl alcohol; octyl dodecanol; myristyl alcohol; isopropyl myristate; isopropyl palmitate; stearic acid; stearyl alcohol; propylene glycol dicaprylate dicaprate (e.g., Miglyol® 812); Softigen® 767; Labrafac™; Lipophile WL 1349; Maisine™ 35-1; Paceol™; glyceryl monooctanoate (e.g., Capmul® MCM; Capmul® PG-8NF) Acconon® CC-400; Captex® 200P; Captex® 300; Captex® 350; polyethylene glycol 400; Capryol™; triacetin; macrogol glycerol hydroxystearate (e.g., Cremophor® RH 40); and others known in the art.

In one embodiment, the lipid or lipophilic vehicle comprises an absorption enhancer. In one aspect, the absorption enhancer also has antioxidant properties. In one aspect, the absorption enhancer comprises d-a-tocopherol polyethylene glycol 1000 succinate (Vitamin E TPGS).

In one embodiment, the lipid or lipophilic vehicle comprises a liquid lipophilic vehicle and a semisolid lipophilic vehicle. In one embodiment, the liquid lipid or lipophilic vehicle comprises olive oil, sunflower oil, canola oil, palmitoleic acid, oleic acid, myristoleic acid, linoleic acid, arachidonic acid, paraffin oil, or mineral oil. In another embodiment, the semi-solid lipophilic vehicle can be a polyethylene glycol glyceride ester, paraffin wax, or bees wax. In another embodiment, the semi-solid lipophilic vehicle is Gelucire® 33/01, Gelucire® 37/02, Gelucire® 39/01, Gelucire® 43/01, Gelucire® 44/14, Gelucire® 50/02, Gelucire® 50/13, Gelucire® 53/10, or Gelucire® 62/02. In one aspect, the liquid lipid or lipophilic vehicle is olive oil.

In one embodiment, the lipid or lipophilic vehicle comprises soybean oil. In another embodiment, the lipid or lipophilic vehicle comprises fish oil. In another embodiment, the lipid or lipophilic vehicle comprises Miglyol® 812. In another embodiment, the lipid or lipophilic vehicle comprises Paceol™. In another embodiment, the lipid or lipophilic vehicle comprises Capryol™. In another embodiment, the lipid or lipophilic vehicle comprises soybean oil.

In another embodiment, the lipid or lipophilic vehicle comprises polyunsaturated fatty acid(s) (PUFA(s)). Suitable PUFAs for the oral pharmaceutical composition described herein comprise fish oils, egg oils, squid oils, krill oils, nut oils, seed oils; soy oils, avocado oils, seabuckthorn seed or berry oils, clary sage seed oils, algal oils, flaxseed oils, sacha ichi oils, echium oils, or hemp oils.

In one embodiment, PUFAs are obtained from fish oils. Suitable fish for extracting fish oil comprise herring, sardines, mackerel (e.g., Spanish, king, Atlantic, or Pacific mackerel), salmon, halibut, tuna, swordfish, greenshell mussels, tilefish, tuna, pollock, cod, catfish, flounder, grouper, mahi mahi, orange roughy, red snapper, shark, hoki, gemfish, blue eye cod, Sydney rock oysters, snapper, or combinations thereof. In one aspect, PUFAs are obtained from fish comprising salmon, herring, mackerel, anchovies, or sardines.

In another embodiment, PUFAs comprise omega-3 fatty acids or polyunsaturated omega-3 fatty acids comprising hexadecatrienoic acid (HTA; all-cis 7,10,13-hexadecatrienoic acid), alpha-linolenic acid (ALA; all-cis-9,12,15-octadecatrienoic acid), stearidonic acid (SDA; all-cis-6,9,12,15,-octadecatetraenoic acid), eicosatrienoic acid (ETE; all-cis-11,14,17-eicosatrienoic acid), eicosatetraenoic acid (ETA; all-cis-8,11,14,17-eicosatetraenoic acid), eicosapentaenoic acid (EPA, timnodonic acid; all-cis-5,8,11,14,17-eicosapentaenoic acid), heneicosapentaenoic acid (HPA; all-cis-6,9,12,15,18-heneicosapentaenoic acid), docosapentaenoic acid (DPA, clupanodonic acid; all-cis-7,10,13,16,19-docosapentaenoic acid), docosahexaenoic acid (DHA, cervonic acid; all-cis-4,7,10,13,16,19-docosahexaenoic acid), tetracosapentaenoic acid (all-cis-9,12,15,18,21-tetracosapentaenoic acid), tetracosahexaenoic acid (nisinic acid; all-cis-6,9,12,15,18,21-tetracosahexaenoic acid), and free acids, ethyl esters, or other esters or salts thereof. In one aspect, the pharmaceutical ingredient comprises a highly purified omega-3 free fatty acid, ester, re-esterified triglyceride, or salt thereof.

In another embodiment, PUFAs comprise omega-6 fatty acids or polyunsaturated omega-6 fatty acids comprising Linoleic acid (LA; all-cis-9,12-octadecadienoic acid), Gamma-linolenic acid (GLA; all-cis-6,9,12-octadecatrienoic acid), Calendic acid (8E,10E,12Z-octadecatrienoic acid), Eicosadienoic acid (all-cis-11,14-eicosadienoic acid), Dihomo-gamma linolenic acid (DGLA; all-cis-8,11,14-eicosatrienoic acid), Arachidonic acid (AA; all-cis-5,8,11,14-eicosatetraenoic acid), Docosadienoic acid (all-cis-13,16-docosadienoic acid), Adrenic acid (all-cis-7,10,13,16-docosatetraenoic acid), Docosapentaenoic acid (osbond acid; all-cis-4,7,10,13,16-docosapentaenoic acid), Tetracosatetraenoic acid (all-cis-9,12,15,18-tetracosatetraenoic acid), Tetracosapentaenoic acid (all-cis-6,9,12,15,18-tetracosapentaenoic acid) and free acids, ethyl esters, or other esters or salts thereof. In one aspect, the pharmaceutical ingredient comprises a highly purified omega-6 fatty acid, ester, re-esterified triglyceride, or salt thereof.

In another embodiment, the matrix comprises a hydrophilic ionic polymer. In one embodiment, the hydrophilic polymers comprise polyhydroxylalkylenediamine, dimethylaminoethyl methacrylate copolymer, Poly(butyl methacrylate-co-(2-dimethylaminoethyl)methacrylate-co-(2-dimethylaminoethyl) 1:2:1 (Eudragit® EPO); sodium carboxy methylcellulose, carboxymethyl cellulose ethylenediamine, sodium alginate, alginic acid, pectin, carbomers, Carbopol® copolymers, such as Carbopol® 934, Carbopol® 940, Carbopol® 941 or Carbopol® 974P; a Pemulen® polymer; polycarbophil poly galacturonic acid, polyglucoronic acid, chondroitic sulfate, carrageenan, and acrylic methacrylate copolymers. In one aspect, the hydrophilic polymer swells in aqueous media. In another aspect, the hydrophilic polymers swell at a pH of about 4 to about 6. In another embodiment, one or more hydrophilic ionic polymers form ionic interactions. In another embodiment, the matrix comprises anionic polymers, cationic polymers, or mixtures thereof. In another embodiment, a hydrophilic cationic polymer and a hydrophilic anionic polymer are combined to form an ionic polymer complex or network.

In another embodiment, the matrix comprises a non-ionic surfactant. The surfactant can have a hydrophilic/lipophilic balance (HLB) value between about 1 and about 25 and a melting point between about 25° C. and about 70° C. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993). Suitable non-ionic surfactants include: Pluronic® 10R5, Pluronic® 17R2, Pluronic® 17R4, Pluronic® 25R2, Pluronic® 25R4, Pluronic® 31R1, Pluronic® F. 108, Pluronic® F. 108 NF, Pluronic® F. 108, Pluronic® F. 108NF, Poloxamer 338, Pluronic® F. 127, Pluronic® F. 127 NF, Pluronic® F. 127 NF 500 BHT Prill, Pluronic® F. 127 NF Prill, Poloxamer 407, Pluronic® F 38, Pluronic® F 38 Pastille, Pluronic® F 68, Pluronic® F 68 LF Pastille, Pluronic® F. 68 NF, Pluronic® F. 68 NF Prill, Poloxamer 188, Pluronic® F. 68 Pastille, Pluronic® F. 77, Pluronic® F. 77 Micropastille, Pluronic® F. 87, Pluronic® F. 87 NF, Pluronic® F. 87 NF Prill, Poloxamer 237, Pluronic® F. 88, Pluronic® F. 88 Pastille, Pluronic® F. 98, Pluronic® L 10, Pluronic® L 101, Pluronic® L 121, Pluronic® L 31, Pluronic® L 35, Pluronic® L 43, Pluronic® L 61, Pluronic® L 62, Pluronic® L 62 LF, Pluronic® L 62D, Pluronic® L 64, Pluronic® L 81, Pluronic® L 92, Pluronic® N 3, Pluronic® P 103, Pluronic® P 104, Pluronic® P 105, Pluronic® P 123 Surfactant, Pluronic® P 65, Pluronic® P 84, Pluronic® P 85, Adogen® 464, Alkanol® 6112, Brij® 52, Brij® 93, Brij® S2, Brij® S, Brij® 58, Brij® C10, Brij® L4, Brij® O10, Brij® O10, BRIJ® O20, Brij® S10, Brij® S20, ethylenediamine tetrakis(ethoxylate-block-propoxylate)tetrol, ethylenediamine tetrakis(ethoxylate-block-propoxylate)tetrol, ethylenediamine tetrakis(propoxylate-block-ethoxylate)tetrol, IGEPAL® CA-210, IGEPAL® CA-520, IGEPAL® CA-720, IGEPAL® CO-520, IGEPAL® CO-630, IGEPAL® CO-720, IGEPAL® CO-890, IGEPAL® DM-970, MERPOL® DA, MERPOL® HCS, MERPOL® OJ, MERPOL® SE, MERPOL® SH, MERPOL® A, Poly(ethylene glycol)sorbitan tetraoleate, poly(ethylene glycol)sorbitol hexaoleate, poly(ethylene glycol) (12), poly(ethylene glycol) (18), polyethylene-block-poly(ethylene glycol), sorbitan monopalmitate, 2,4,7,9-tetramethyl-5-decyne-4,7-diol ethoxylate, Nonidet™ P-40, Triton™ N-101, Triton™ X-100, Triton™ X-114, Triton™ X-405, TWEEN® (e.g., TWEEN® 20, TWEEN® 40, TWEEN® 60, TWEEN® 80), Zonyl® FS-300, or Zonyl® FSN. In one embodiment, the non-ionic surfactant comprises TWEEN® (e.g., TWEEN® 20, TWEEN® 40, TWEEN® 60, TWEEN® 80), Span® (e.g., Span 20, Span® 40, Span® 80), lecithin, Labrasol®, Lauroglycol™, Geleol™, macrogol glycerol hydroxystearate (e.g., Cremophor® RH 40); or Plurol® Oleique cc497.

In one embodiment, the non-ionic surfactant comprises Cremophor® RH 40. In another embodiment, the non-ionic surfactant comprises Cremophor® RH 40. In another embodiment, the non-ionic surfactant comprises TWEEN® 80. In another embodiment, the non-ionic surfactant comprises Span® 80. In another embodiment, the non-ionic surfactant comprises Labrasol®. In another embodiment, the non-ionic surfactant comprises Lauroglycol™. In another embodiment, the non-ionic surfactant comprises lecithin.

In another embodiment, the matrix comprises a hygroscopic polymer. In one embodiment, the hygroscopic polymers include polyvinylpyrrolidone, copovidone, hydroxypropylmethylcellulose, hydroxypropylcellulose, ethyl cellulose, methylcellulose, and polyethylene oxide. Suitable hygroscopic polymers include polyvinyl alcohol, a copolymer of polyvinylpyrrolidone and polyvinyl acetate, hydroxypropyl cellulose, hydroxypropyl methylcellulose, hydroxyethyl cellulose, hydroxymethyl cellulose, gelatin, polyethylene oxide, acacia, dextrin, starch, poly hydroxyethylmethacrylate, a water-soluble non-ionic polymethacrylate or copolymer thereof, a modified cellulose, a modified polysaccharide, a non-ionic gum, or a non-ionic polysaccharide.

In another embodiment, the matrix comprises a pH buffering agent. Suitable pharmaceutically acceptable buffering agents comprise arginine, aminomethyl propanol, tetrahydroxypropyl ethylenediamine, triethanolamine, tromethamine, PEG-15 cocamine, di-isopropanol amine, tri-isopropanol amine, N-methyl-D-glucamine, glycine, malate, tartarate, lactate, citrate, acetate, sodium bicarbonate, sodium phosphate, or other buffering agents, having pKₐs at any physiologically acceptable pH, generally from about pH 4 to about pH 7. Amino acids or other physiological metabolites may be used as buffering agents. A combination of buffering agents may also be employed, such as phosphate and acetate, and the like.

In another embodiment, the matrix comprises a neutralizing agent. Suitable pharmaceutically acceptable neutralizing agents comprise HCl, phosphoric acid, carbonic acid, sodium hydroxide, ammonium hydroxide, potassium hydroxide, sodium bicarbonate, sodium carbonate, and the like.

In one embodiment, one or more antioxidants can be present in the matrix fills described herein. Suitable antioxidants comprise tocopherols (e.g., alpha-tocopherol, beta-tocopherol, gamma-tocopherol, or delta-tocopherol), butylated hydroxytoluene (BHT), butylated hydroxyanisole (BHA), citric acid, ascorbic acid, phenolic diterpenes (e.g., carnosic acid, carnosol, rosmanol, epirosmanol, isorosmanol, or methyl carnosate), rosmarinic acid, eugenol, eugenyl acetate, clove bud extract, methanolic extract, tea catechins (e.g., epigallocatechin gallate, epicatechin gallate, epigallocatechin, or epicatechin), or any mixture thereof. In one aspect, the one or more antioxidants are present in the matrix fills described herein comprise from about 0.001% to about 2%, by weight of the matrix fill including all integers within the specified range.

In one embodiment, the matrix optionally comprises a water-binding excipient. Suitable water-binding excipients function as dessicants, which reduce or prevent water from reaching the active pharmaceutical ingredient. Exemplary water-binding excipients comprise cyclodextrin (e.g., α-cyclodextrin, β-cyclodextrin, γ-cyclodextrin) Aerosil® 200, and Syloid® FP Silica.

In another embodiment, the matrix comprises a hydrophilic internal phase and a lipid or lipophilic external phase. The internal phase can also be structured. A “structured” internal phase, as used herein, means a solid, semisolid, or a gel whose shape is relatively stable and does not usually aggregate to form a large globule. A structured internal phase therefore provides controlled drug release and stabilizes the physical state of the matrix. Without being bound to any theory, it is believed that the structured nature of the matrix impedes solvation and/or diffusion of the active pharmaceutical ingredient out of the matrix.

In one embodiment, the active pharmaceutical ingredient can be dispersed in the internal phase as a suspension form. In another embodiment, the active pharmaceutical ingredient can be in solution with the internal phase. In another embodiment, the active pharmaceutical ingredient can be dispersed in the internal phase as a solid form.

In one embodiment, the lipid or lipophilic vehicle comprises a liquid lipid or lipophilic vehicle, a semisolid lipid or lipophilic vehicle, or a mixture thereof.

In one embodiment, the lipid or lipophilic vehicle comprises from about 40% to about 90% of the total matrix mass including all integers within the specified range. In another embodiment, the lipid or lipophilic vehicle comprises from about 60% to about 80% of the total matrix mass including all integers within the specified range. In one aspect, the lipid or lipophilic vehicle is from about 70% to about 90% of the total matrix mass, including all integers within the specified range. In another aspect, the lipid or lipophilic vehicle is about 81% of the total matrix mass. In one aspect, the lipid or lipophilic vehicle is about 90% of the total matrix mass.

In one embodiment, the non-ionic surfactant comprises from about 5% to about 20% of the total matrix mass including all integers within the specified range. In another embodiment, the non-ionic surfactant comprises from about 5% to about 15% of the total matrix mass including all integers within the specified range. In one aspect, the non-ionic surfactant comprises about 10% of the total matrix mass. In another aspect, the non-ionic surfactant comprises about 15% of the total matrix mass. In another aspect, the non-ionic surfactant comprises about 20% of the total matrix mass.

In one embodiment, the lipid or lipophilic vehicle and non-ionic surfactant comprises from about 50% to about 90% of the total matrix mass including all integers within the specified range. In one aspect, the lipid or lipophilic vehicle and non-ionic surfactant comprises about 60% of the total matrix mass including all integers within the specified range. In another aspect, the lipid or lipophilic vehicle and non-ionic surfactant comprises about 72% of the total matrix mass. In another aspect, the lipid or lipophilic vehicle and non-ionic surfactant comprises about 80% of the total matrix mass. In another aspect, the lipid or lipophilic vehicle and non-ionic surfactant comprises about 85% of the total matrix mass.

In one embodiment, one or more pharmaceutically acceptable excipients comprise from about 0.01% to about 15% of the total matrix mass including all integers within the specified range. In one aspect, one or more pharmaceutically acceptable excipients comprise about 0.01% of the total matrix mass. In another aspect, one or more pharmaceutically acceptable excipients comprise about 4% of the total matrix mass. In another aspect, one or more pharmaceutically acceptable excipients comprise about 5% of the total matrix mass. In another aspect, one or more pharmaceutically acceptable excipients comprise about 6% of the total matrix mass. In another aspect, one or more pharmaceutically acceptable excipients comprise about 7% of the total matrix mass. In another aspect, one or more pharmaceutically acceptable excipients comprise about 10% of the total matrix mass.

In one embodiment, the water-binding excipient comprises from about 1% to about 7% of the total matrix mass including all integers within the specified range. In one aspect, the water binding excipient comprises about 1% of the total matrix mass. In another aspect, the water binding excipient comprises about 3% of the total matrix mass. In another aspect, the water binding excipient comprises about 5% of the total matrix mass. In another aspect, the water binding excipient comprises about 7% of the total matrix mass.

In one embodiment, the absorption enhancer comprises from about 1% to about 7% of the total matrix mass including all integers within the specified range. In one aspect, the absorption enhancer comprises about 1% of the total matrix mass. In another aspect, the absorption enhancer comprises about 3% of the total matrix mass. In another aspect, the absorption enhancer comprises about 5% of the total matrix mass. In another aspect, the absorption enhancer comprises about 7% of the total matrix mass.

In another embodiment, the weight percentage ratio of the lipid or lipophilic vehicle to the non-ionic surfactant in the matrix fill comprises from about 1:1 to about 9:1 including all iterations of ratios within the specified range. In one aspect, the ratio of the lipid or lipophilic vehicle to the non-ionic surfactant is about 1:1. In another aspect, the ratio of the lipid or lipophilic vehicle to the non-ionic surfactant is about 3:1. In another aspect, the ratio of the lipid or lipophilic vehicle to the non-ionic surfactant is about 4:1. In another aspect, the ratio of the lipid or lipophilic vehicle to the non-ionic surfactant is about 5:1. In another aspect, the ratio of the lipid or lipophilic vehicle to the non-ionic surfactant is about 6:1. In another aspect, the ratio of the lipid or lipophilic vehicle to the non-ionic surfactant is about 7:1.

In another embodiment, the weight percentage ratio of the active pharmaceutical ingredient to the lipid or lipophilic vehicle in the matrix fill comprises from about 1:10 to about 1:1 including all iterations of ratios within the specified range. In one aspect, the ratio of the active pharmaceutical ingredient to the lipid or lipophilic vehicle is about 1:9. In another aspect, the ratio of the active pharmaceutical ingredient to the lipid or lipophilic vehicle is about 1:7. In another aspect, the ratio of the active pharmaceutical ingredient to the lipid or lipophilic vehicle is about 1:5. In another aspect, the ratio of the active pharmaceutical ingredient to the lipid or lipophilic vehicle is about 1:3. In another aspect, the ratio of the active pharmaceutical ingredient to the lipid or lipophilic vehicle is about 1:2. In another aspect, the ratio of the active pharmaceutical ingredient to the lipid or lipophilic vehicle is about 1:1.

In another embodiment, the weight percentage ratio of the active pharmaceutical ingredient to the non-ionic surfactant in the matrix fill comprises from about 1:10 to about 3:1 including all iterations of ratios within the specified range. In one aspect, the ratio of the active pharmaceutical ingredient to the non-ionic surfactant is about 1:9. In another aspect, the ratio of the active pharmaceutical ingredient to the non-ionic surfactant is about 1:5. In another aspect, the ratio of the active pharmaceutical ingredient to the non-ionic surfactant is about 1:3.5. In another aspect, the ratio of the active pharmaceutical ingredient to the non-ionic surfactant is about 1:1. In another aspect, the ratio of the active pharmaceutical ingredient to the non-ionic surfactant is about 2:1. In another aspect, the ratio of the active pharmaceutical ingredient to the non-ionic surfactant is about 2.5:1.

In another embodiment, the ratio of the total matrix to the active pharmaceutical ingredient comprises from about 1:1 to about 99:1, including all iterations of ratios within the specified range. In another embodiment, the ratio of the total matrix to the active pharmaceutical ingredient comprises from about 1:1 to about 20:1, including all iterations of ratios within the specified range. In one aspect, the ratio of the matrix to the active pharmaceutical ingredient is about 2.5:1. In another aspect, the ratio of the matrix to the active pharmaceutical ingredient is about 5:1. In another aspect, the ratio of the matrix to the active pharmaceutical ingredient is about 9:1. In another aspect, the ratio of the matrix to the active pharmaceutical ingredient is about 19:1.

In one embodiment described herein, the pharmaceutical composition comprises a soft capsule shell comprising a matrix comprising an active pharmaceutical ingredient.

In one embodiment described herein, the active pharmaceutical ingredient comprises a moisture- and/or light-sensitive active pharmaceutical ingredient. In one aspect, the moisture- and/or light-sensitive active pharmaceutical ingredient comprises at least one of: Acarbose (Precose); Acyclovir (Zovirax); Alendronate sodium and cholecalciferol (Fosamax plus D); Alfuzosin HCl (Uroxatral); Aliskiren (Tekturna); Alosetron (Lotronex); Alprazolam (Xanax); Amantadine hydrochloride (Symmetrel); Amiloride hydrochloride (Midamor); Amiloride/hydrochlorothiazide (Moduretic); AmLodipine and benazepril hydrochloride (Lotrel); AmLodipine and valsartan (Exforge); Aspirin and dipyridamole (Aggrenox); Atropine sulfate, hyoscyamine hydrobromide, phenobarbital, scopolamine hydrobromide (Donnatal); Azathioprine (Azasan); Benazepril hydrochloride (Lotensin); Benazepril hydrochloride and hydrochlorothiazide (Lotensin HCT); Benzonatate (Tessalon); Biphosphonate combination (Fosamax Plus D); Bisoprolol fumarate (Zebeta); Bupropion hydrochlroide (Wellbutrin); Calcium lactate (Cal-Lac); Captopril (Capoten); Captopril and hydrochlorthiazide (Capozide); Carbamazepine (Epitol, Tegretol); Carbamazepine extended release (Carbatrol, Equetro); Carvedilol (Coreg); Cefditoren pivoxil (Spectracef); Cefpodoxime (Vantin); Chloroquine phosphate (Aralen Phosphate); Clomiphene Citrate (Clomid, Milophene, Serophene); Clomipramine hydrochloride (Anafranil); Clonazepam (Klonopin); Clonidine hydrochloride and Chlorthalidone (Clorpres); Clorazepate dipotassium (Tranxene); Clozapine (Clozaril, FazaClo); Codeine phosphate and codeine sulfate (Codeine); Colesevelam hydrochloride (WelChol); Cortisone (Cortisone); Cysteamine bitartrate (Cystagon); Deferasirox (Exjade); Desloratadine and pseudoephedrine (Clarinex-D); Dexmethylphenidate hydrochloride (Focalin); Diazepam (Valium); Diclofenac potassium (Voltaren); Diclofenac sodium (Cataflam, Voltaren); Dicyclomine hydrochloride (Bentyl, Byclomine); Dabigatran etexilate mesylate (Pradaxa); Diltiazem (Cardizem, Cartia, Diltia, Taztia, Tiazac, Dilacor); Dofetilide (Tikosyn); Enalapril maleate Vasotec); Enalapril maleate and felodipine (Lexxel); Erythromycin base (Ery-Tab, PCE Dispertab, Eryc); Erythromycin ethylsuccinate (E.E.S.); Etodolac (Etodolac); Etravirine (Intelence); Ezetimibe (Zetia); Famotidine (Pepcid); Fenofibrate (Tricor, Triglide, Lofibra, Antara); Fentanyl citrate (Fentora); Ferrous fumarate (Hemocyte, Ferretts, Nephro-Fer, Ferro-Sequels); Finasteride (Propecia, Proscar); Flavocoxid (Limbrel); Folic acid (Deplin, Folvite); Fosinopril sodium (Monopril); Frovatriptan succinate (Frova); Glipizide (Glucotrol); Hydrochlorothiazide (HydroDIURIL, Hydro-Par, Ezide, Microzide); Imatinib (Gleevec); Indinavir sulfate (Crixivan); Indomethacin (Indocin); Isoniazid Nydrazid); Isosorbide mononitrate (Monoket, Imdur); Isradipine (DynaCirc); Itraconazole (Sporanox); Ketoconazole (Nizoral); Ketoprofen (Ketoprofen); Labetalol hydrochloride (Normodyne, Trandate); Lansoprazole (Prevacid); Lansoprazole, amoxicillin and clarithromycin (Prevpac); Lanthanum carbonate (Fosrenol); Levodopa and carbidopa (Sinemet, Parcopa); Levothyroxine sodium (Synthroid); L-Hyoscyamine sulfate (Anaspaz, ED-SPAZ, Levsin, Symax, Neosol, Mar-Spas, Levsinex); Linezolid (Zyvox); Lisinopril (Prinivil, Zestril); Lisinopril/hydrochlorothiazide (Tenoretic); Lithium (Lithobid); Mefenamic acid (Ponstel); Metaproterenol sulfate (Alupent); Metyrapone (Metopirone); Metformin (Fortamet, Glucophage); Methocarbamol (Robaxin); Methsuximide (Celontin); Methylphenidate (Concerta, Ritalin); Metoprolol tartrate (Lopressor); Minocycline (Solodyn); Mirtazapine (Remeron); Moexipril (Univasc); Montelukast (Singulair); Morphine (Kadian, MS Contin); Mycophenolate (Myfortic, CellCept); Naproxen and lansoprazole (Prevacid Naprapac); Nifedipine (Procardia); Nisoldipine (Sular); Nitroglycerin (Nitroquick); Olanzapine (Zyprexa); Olanzapine/fluoxetine hydrochloride (Symbyax); Omeprazole (Prilosec); Orlistat (Xenical); Oxybutynin chloride (Ditropan); Paliperidone (Invega); Pancrelipase (Creon); Paromomycin sulfate (Humatin); Perindopril (Aceon); Phendimetrazine tartrate (Bontril); Phenytoin (Phenytek); Pioglitazone (Actos); Pioglitazone/glimepiride (Duetact); Pioglitazone/metformin hydrochloride (Actoplus Met); Pravastatin (Pravachol); Prazosin hydrochloride (Minipress); Procainamide hydrochloride (Pronestyl); Progesterone (Prometrium); Propranolol (Inderal); Propylthiouracil(PTU) (Propylthiouracil(PTU)); Quazepam (Doral); Rabeprazole sodium (Aciphex); Ramelteon (Rozerem); Repaglinide (Prandin); Risedronate sodium with calcium carbonate (Actonel with Calcium); Risperidone (Risperdal); Rosuvastatin calcium (Crestor); Sapropterin dihydrochloride (Kuvan); Sevelamer (Renegal); Sibutramine hydrochloride (Meridia); Tacrine hydrochloride (Cognex); Terazosin (Hytrin); Tiagabine hydrochloride (Gabitril); Topiramate (Topamax); Valsartan (Diovan); Valsartan/hydrochlorothiazide (Diovan HCT); Zafirlukast (Accolate); Zidovudine (Retrovir); or Zolmitriptan (Zomig).

In another embodiment described herein, the active pharmaceutical ingredient comprises a light-sensitive active pharmaceutical ingredient. In one aspect, the light-sensitive active pharmaceutical ingredient comprises at least one of: Acebutolol HCl (Sectral); Acetaminophen and codeine phosphate (Tylenol with codeine); Acetaminophen and hydrocodone bitartrate (Vicodin, Zydone); Acetazolamide (Diamox Sequels); Acitretin (Soriatane); Allopurinol (Zyloprim, Aloprim); Aminoglutethimide Cytadren); Amiodarone (Cordarone, Pacerone); Amitriptyline (Elavil); Amlodipine (Norvasc, Amvaz); Amphetamine and dextroamphetamine salts (Adderall XR); Anagrelide HCl (Agrylin); Atenolol (Tenormin); Auranofin (Ridaura); Bexarotene (Targretin); Biperiden (Akineton); Bromocriptine mesylate (Parlodel); Bumetanide (Bumex); Buspirone (Buspar); Calcitriol (Rocaltrol); Carbinoxamine (Palgic, Histex Conn.); Chlordiazepoxide (Librium); Chlorpromazine (Thorazine); Chlorzoxazone (Paraflex , Remular-S, Parafon Forte DSC); Cimetidine (Tagamet); Clarithromycin (Biaxin); Clonidine (Catapres); Cyclizine (Bonine, Marezine); Cyclobenzaprine hydrochloride (Flexeril, Fexmid, Amrix); Dapsone (Dapsone); Darifenacin (Enablex); Desloratadine (Clarinex); Dextroamphetamine (DextroStat); Diazoxide (Proglycem); Digoxin (Lanoxin, Digitek); Dolasetron (Anzemet); Doxycycline (Alodox, Periostat, Adoxa, Vibra-Tabs, Doryx, Monodox, Vibramycin); Entecavir (Baraclude); Ergoloid mesylates (Ergoloid mesylates); Ergonovine maleate (Ergotrate); Ergotamine (Ergomar); Estradiol (Femtrace, Gynodiol, Estrace); Estropipate (Ogen); Ethosuximide (Zarontin); Ezetimibe/simvastatin (Vytorin); Felodipine (Plendil); Fluoxetine (Prozac); Flurazepam hydrochloride (Fluphenazine); Flutamide (Flutamide); Fluvastatin (Lescol); Furosemide (Lasix); Gemifloxacin (Factive); Glyburide/metformin (Glucovance); Granisetron (Kytril); Griseofulvin ultramicrosize (Gris-PEG); Guanfacine (Tenex); Haloperidol (Haldol); Homatropine hydrobromide, hydrocodone bitartrate (Hycodan); Hydralazine HCl (Apresoline); Hydrocodone and ibuprofen (Vicoprofen); Hydromorphone (Dilaudid); Hydroxychloroquine sulfate (Plaquenil); Hydroxyzine (Vistaril); Isosorbide dinitrate (Isordil Titradose, Isochron, Dilatrate); Isotretinoin (Accutane, Amnesteem, Claravis, Sotret); Lamotrigine (Lamictal); Leflunomide (Arava); Leucovorin calcium (Leucovorin); Liotrix (Thyrolar); Lisdexamfetamine (Vyvanse); Lorazepam (Ativan); Losartan potassium (Cozaar); Lovastatin (Mevacor); Magnesium salicylate (Novasal); Maprotiline (Maprotiline); Meclizine (Antivert); Mephalan (Alkeran); Methadone (Dolophine); Methamphetamine hydrochloride (Desoxyn); Methenamine hippurate (Hiprex); Methotrexate (Methotrexate); Methylergonovine maleate (Methergine); Metoclopramide (Reglan); Metolazone (Zaroxolyn); Metronidazole (Flagyl); Molindone hydrochloride (Moban); Moricizine (Ethmozine); Nadolol (Corgard); Naltrexone hydrochloride (ReVia); Naproxen (Anaprox); Nebivolol (Bystolic); Nefazodone (Nefazodone); Nilutamide (Nilandron); Nimodipine (Nimotop); Nizatidine (Axid); Nortriptyline (Pamelor); Nystatin (Mycostatin); Ondansetron (Zofran); Oxaprozin (Daypro); Oxycodone (Oxycontin); Oxycodone/acetaminophen (Percocet); Oxycodone/aspirin (Percodan); Pantoprazole sodium (Protonix); Papaverine HCl (Papaverine HCl); Penbutolol Sulfate (Levatol); Perphenazine (Perphenazine); Pimozide (Orap); Pindolol (Visken); Pramipexole dihydro chloride (Mirap ex); Prednisolone (Prednisolone); Prochlorperazine (Compazine); Promethazine (Phenergan); Propantheline bromide (Pro-Banthine); Pyrimethamine (Daraprim); Quinapril (Accupril); Quinidine Sulfate (Qualaquin); Riluzole (Rilutek); Ritonavir (Norvir); Ropinirole hydrochloride (Requip); Rosiglitazone (Avandia); Rosiglitazone/glimiperide (Avandaryl); Rosiglitazone/metformin (Avandamet); Selegiline (Eldepryl, Zelapar); Sirolimus (Rapamune); Sotalol hydrochloride (Betapace); Spironolactone (Aldactone); Stavudine (Zerit); Sulfadiazine (Sulfadiazine); Tamoxifen citrate (Nolvadex); Temazepam (Restoril); Terbinafine (Lamisil); Terbutaline (Brethine); Tetracycline hydrochloride (Sumycin); Thalidomide (Thalomid); Theophylline (Theochron); Thioridazine (Thioridazine); Thiothixene (Thioxene); Thyroid (Armour thyroid); Timolol maleate (Timolol); Tinidazole (Tindamax); Tolmetin (Tolmetin); Tolterodine (Detrol LA); Topotecan (Hycamtin); Toremifene citrate (Fareston); Trazodone (Desyrel); Tretinoin (Vesanoid); Triamterene (Dyrenium); Triamterene/hydrochlorothiazide (Dyazide); Trimethobenzamide (Tigan); Trimethoprim hydrochloride (Proloprim); Vitamin K (Mephyton); Warfarin Sodium (Coumadin); Zaleplon (Sonata); Zileuton (Zyflo); Ziprasidone (Geodon); or Zonisamide (Zonigran).

In another embodiment, any of the above mentioned active drug substances may also be in the form of pharmaceutically acceptable salts, uncharged or charged molecules, molecular complexes, solvates, or anhydrates thereof, and, if relevant, single isomers, enantiomers, racemic mixtures, or mixtures thereof.

In another embodiment, the pharmaceutical compositions described herein may comprise pharmaceutically acceptable salts of any of the above mentioned active drug substances.

The term “pharmaceutically acceptable salts” of an active drug substance includes alkali metal salts such as, for example, sodium or potassium salts, alkaline earth metal salts such as, for example, calcium and magnesium salts, and salts with organic or inorganic acid such as, for example, hydrochloric acid, hydrobromic acid, nitric acid, sulfuric acid, phosphoric acid, citric acid, formic acid, maleic acid, succinic acid, tartaric acid, methanesulphonic acid, toluenesulphonic acid etc. In another embodiment, pharmaceutically acceptable opioid salts can comprise sulphate salts, hydrochloride salts, and bitartrate salts.

In another embodiment, the active pharmaceutical ingredient may be in any of its crystalline, polymorphous, semi-crystalline, amorphous or poly amorphous forms or mixtures thereof.

The concentration of the active drug substance in the pharmaceutical composition for use according to the disclosure depends on the specific active drug substance, the disease to be treated, the condition of the patient, the age, and gender of the patient, etc. The above-mentioned active drug substances may be known active drug substances and a person skilled in the art will be able to find information as to the dosage of each active drug substance and, accordingly, will know how to determine the amount of each active drug substance in the pharmaceutical composition.

The active pharmaceutical ingredient may be a new chemical entity for which the amount of information is limited. In such cases, the dosage has to be evaluated based on available preclinical and/or clinical data.

In one embodiment, the active pharmaceutical ingredient is sensitive to moisture and/or light. Active pharmaceutical ingredients sensitive to moisture will undergo hydrolytic degradation and active pharmaceutical ingredients sensitive to light will undergo photodegradation. Both hydrolytic degradation and photodegradation reduce the efficacy or activity of the light and/or moisture sensitive active pharmaceutical ingredient. In one aspect, the active pharmaceutical ingredient is sensitive to moisture.

In one embodiment, the pharmaceutical composition described herein, reduces the moisture content or water in contact with the active pharmaceutical ingredient. In one aspect, the matrix fill reduces the migration of water from the capsule shell to the active pharmaceutical ingredient. In another aspect, the matrix fill reduces the hydrolytic degradation of the active pharmaceutical ingredient. Without being bound by any theory, it is thought that the intermolecular forces between the matrix fill comprising the active pharmaceutical ingredient and water result in water being repelled from the matrix fill, while being retained within the soft capsule shell. This effect prevents the soft capsule shell from becoming dry and brittle, while also preventing water from the capsule shell or from the environment from interacting with the active pharmaceutical ingredient.

In another embodiment, the moisture content of the matrix fill after encapsulation in the soft capsule shell described herein is less than 2% of the total matrix fill mass. In another embodiment, the moisture content of the matrix fill after encapsulation in the soft capsule shell described herein is less than 1.5% of the total matrix fill mass. In another embodiment, the moisture content of the matrix fill after encapsulation in the soft capsule shell described herein is less than 1% of the total matrix fill mass. In another embodiment, the moisture content of the matrix fill after encapsulation in the soft capsule shell described herein is less than 0.5% of the total matrix fill mass. In another embodiment, the moisture content of the matrix fill after encapsulation in the soft capsule shell described herein is less than 0.1% of the total matrix fill mass. In another embodiment, the moisture content of the matrix fill after encapsulation in the soft capsule shell described herein is less than 0.05% of the total matrix fill mass. In another embodiment, the matrix fill after encapsulation in the soft capsule shell described herein comprises essentially no moisture or water (e.g., less than 0.03% moisture or water).

In another embodiment, the percent change in moisture content of the matrix fill described herein after storage of the pharmaceutical composition described herein comprising the matrix fills stored in a sealed or opened container (e.g., a bottle closed or opened) for about 24 hours to about 3 months at a temperature from about 30° C. to about 40° C. and at a relative humidity of at least about 60% comprises from about 0.06% to about 5%. In one aspect, the percent change in moisture content of the matrix fill is less than about 4%. In another aspect, the percent change in moisture content of the matrix fill is less than about 2.5%. In another aspect, the percent change in moisture content of the matrix fill is less than about 1.0%. In another aspect, the percent change in moisture content of the matrix fill is less than about 0.5%, 0.1%, about 0.07%, or 0.05%. In another aspect, there is essentially no percent change in moisture content (e.g., less than a 0.03% change) of the matrix fill.

In another embodiment, the percent of active pharmaceutical ingredient degradation products or other impurities in the matrix fills described herein, (e.g., from hydrolytic cleavage, enantiomeric conversion, photodegradation, and/or contamination) after storage of the pharmaceutical composition described herein comprising the matrix fills described herein in a sealed or opened container (e.g., a bottle closed or opened) for about 24 hours to about 3 months at a temperature from about 30° C. to about 40° C. and at a relative humidity of at least about 60% is less than about 3%. In another aspect, the percent of active pharmaceutical ingredient degradation products or other impurities in the matrix fill is less than about 2%. In another aspect, the percent of active pharmaceutical ingredient degradation products or other impurities in the matrix fill is less than about 1%. In another aspect, the percent of active pharmaceutical ingredient degradation products or other impurities in the matrix fill is less than about 0.5%. In another aspect, the percent of active pharmaceutical ingredient degradation products or other impurities in the matrix fill is less than about 0.3%.

In another embodiment described herein, are methods of manufacturing the immediate release matrix fill composition described herein. In one aspect, the lipid or lipophilic vehicle is added to a vessel of a suitable size. Next, one or more pharmaceutically acceptable excipients is added to said vessel and dissolved at about room temperature with an agitator rotating at about 50 RPM to about 1000 RPM until a clear solution is formed. Following the formation of a clear solution step, the active pharmaceutical ingredient is added in an appropriate amount for the desired dosages as described herein and homogenized at about room temperature at about 50 RPM to about 1000 RPM until a homogenous suspension is formed (e.g., suspension free of lumps). Following the mixing step and homogenization step, the mixture comprising the active pharmaceutical ingredient is de-aerated until substantially free of air (e.g., no air bubbles or other sign of air is observable). Following the de-aeration step, the matrix fill composition as described herein is mixed with an agitator rotating at about 50 RPM to about 1000 RPM and is encapsulated in the soft capsule shells described herein using rotary die encapsulation technology as described herein.

In one embodiment described herein, the active pharmaceutical ingredient comprises a moisture-sensitive active pharmaceutical ingredient. In another aspect described herein, the moisture-sensitive active pharmaceutical ingredient is dabigatran etexilate mesylate (Pradaxa), aspirin, or diclofenac. In one aspect, the moisture-sensitive active pharmaceutical ingredient is dabigatran. In another aspect, the moisture-sensitive active pharmaceutical ingredient is dabigatran etexilate mesylate. In another aspect, the moisture-sensitive active pharmaceutical ingredient is aspirin. In another aspect, the moisture-sensitive active pharmaceutical ingredient is diclofenac. In another aspect, the moisture-sensitive active pharmaceutical ingredient is in free acid form or a pharmaceutically acceptable salt form thereof.

In one embodiment described herein, the pharmaceutical composition comprises a soft capsule shell comprising a matrix comprising an active pharmaceutical ingredient.

In one embodiment described herein, the soft capsule shell has the composition of Table 2, including all possible iterations of the specified ranges that provide 100% for the total weight percentage, including or excluding the optional colorings, flavorings, or excipients.

TABLE 2
Exemplary Soft Capsule Shell Composition

| Component | Exemplary Component | Composition Range (%) |
|---|---|---|
| Film-forming polymer | Gelatin | 25-50 |
| Plasticizer | Glycerol | 15-25 |
| Solvent | Water | 20-40 |
| Opacifier (optional) | Titanium dioxide | 0.5-1.5 |
| Coloring agent | Various | 0.05-0.1 |
| (optional) | | |

Film-forming polymers that are useful for creating soft capsules as described herein comprise gelatin or hydroxypropylmethylcellulose (HPMC). In one aspect, the film-forming polymer is gelatin.

Gelatin compositions that are useful for creating enteric soft capsules described herein can be classified as either Type A or Type B gelatin. Examples of gelatin compositions that are useful for creating enteric soft capsule shells as described herein comprise acid bone gelatin, pig skin gelatin, chicken skin gelatin, fish gelatin, acid hide gelatin, gelatin hydrolysate, lime bone gelatin, or combinations thereof. Type A gelatin is derived from the acid hydrolysis of collagen (e.g., acid bone gelatin or pig skin gelatin), while Type B gelatin (e.g., lime bone gelatin) is derived from the alkaline hydrolysis of collagen. Traditionally, bovine bones and skins have been used as raw materials for manufacturing Type A and Type B gelatin while porcine skins have been used extensively for manufacturing Type A gelatin. In general, acid processed gelatins form stronger gels than lime-processed gelatins of the same average molecular weight. In addition, at neutral pH values, Type A gelatins (acid processed gelatins) are typically net cationic and Type B gelatins (alkali processed gelatins) are typically net anionic. The strength of said gelatin compositions are often defined by their Bloom strength or grade in the range of about 30 Bloom to about 400 Bloom.

Plasticizers that are useful for creating soft capsules as described herein are glycerol, sorbitol, polyethylene glycols, or combinations thereof. The weight ratio between the film-forming polymer, plasticizer, and solvent is adjusted so that the gel mass is flowable and not too viscous, and can be made into soft capsules using rotary die encapsulation methods.

In one embodiment, the enteric soft capsule shell has the exemplary composition shown in Table 3.

TABLE 3
Exemplary Soft Capsule Shell Composition

| Component | Percent weight (%) |
|---|---|
| Gelatin | 43 |
| Glycerol | 20 |
| Titanium dioxide | 0.7 |
| Coloring agent | 0.1 |
| Water | 36.2 |
| TOTAL | 100% |
| Final pH | ~4-7 |
| Ratio total plasticizer to gelatin | 20:43 (0.46:1) |
| Water content in dried soft capsule shell: | 8-15 |

In one embodiment described herein, the soft capsule comprises about 43% of at least one film-forming polymer; about 20% of at least one plasticizer; about 36% water; optionally, about 0.7% titanium dioxide; and optionally, about 0.1% of at least one coloring agent.

In one embodiment, the weight percentage range of film-forming polymer of the soft capsule described herein comprises from about 35% to about 45%, including all integers within the specified range. In one aspect, the film-forming polymer weight percentage is about 38%. In another aspect, the film-forming 1 polymer weight percentage is about 42%. In another aspect, the film-forming polymer weight percentage is about 44%.

In one embodiment, the weight percentage range of plasticizer comprises from about 15% to about 22%, including all iterations of integers with the specified range. In one aspect, the plasticizer weight percentage is about 17%. In another aspect, the plasticizer weight percentage is about 18.5%. In another aspect, the plasticizer weight percentage is about 20%.

In one embodiment, the weight percentage ratio range of plasticizer to film-forming polymer comprises from about 0.33:1 to about 0.56:1, including all iterations of iterations of ratios with the specified range. In one embodiment, the weight percentage ratio range of plasticizer to film-forming polymer is about 0.38:1. In one embodiment, the weight percentage ratio range of plasticizer to film-forming polymer is about 0.42:1. In one embodiment, the weight percentage ratio range of plasticizer to film-forming polymer is about 0.46:1. In one embodiment, the weight percentage ratio range of plasticizer to film-forming polymer is about 0.52:1.

In one aspect, soft capsules are made using a rotary die apparatus as described in U.S. Pat. Nos. 5,459,983; 5,146,730; and 6,482,516, each of which are incorporated by reference herein for such teachings.

Another embodiment described herein includes a process of manufacturing soft capsules comprising the pharmaceutical composition as described herein. The process includes preparing a gel mass composition comprising a film-forming, water-soluble polymer, an appropriate plasticizer, and solvent; casting the gel mass into films or ribbons using heat-controlled drums or surfaces; and manufacturing a soft capsule comprising a matrix fill using rotary die technology. The thickness of the films or ribbons that form the soft capsule shell comprises from about 0.010 inches (≈0.254 mm) to about 0.050 inches (≈21.27 mm), including all integers within the specified range. The shell thickness can be about 0.010 inch (≈0.254 mm), about 0.015 inch (≈0.381 mm), about 0.02 in (≈0.508 mm), about 0.03 in (≈0.762 mm), about 0.04 in (≈21.02 mm), or about 0.05 in (≈21.27 mm). In one embodiment, the thickness comprises from about 0.02 inches (≈0.508 mm) to about 0.040 inches (≈21.02 mm). In one embodiment, the shell thickness is about 0.028 inches (≈0.711 mm). In another embodiment, the shell thickness is about 0.033 inches (≈0.838 mm). In another embodiment, the shell thickness is about 0.038 inches (≈0.965 mm).

The pharmaceutical composition described herein can comprise a soft capsule comprising a matrix fill that is liquid, semi-solid, or solid. Capsules prepared as described herein can contain a hydrophobic solution or suspension, such as vegetable oils or shortening, or waxes, or combinations thereof. The matrix can be formulated to prevent interaction with the capsule shell components and release the pharmaceutical composition at a specified rate.

In another embodiment described herein, the pharmaceutical composition comprises an enteric soft capsule shell. Enteric soft capsules are described in International Patent Application Publication No. WO 2004/030658 and U.S. Patent Application Publication No. US 2006/0165778, both of which are incorporated by reference herein for such teachings. The enteric soft capsule shell can comprise one or more film forming polymers, one or more enteric acid insoluble polymers, one or more plasticizers, one or more alkali neutralizing agents, one or more solvents, optionally one or more colorants, and optionally one or more flavorings and/or other conventionally accepted pharmaceutical excipients or additives.

Film-former polymers that are useful for creating enteric soft capsules are gelatin or hydroxypropylmethylcellulose (HPMC). In one aspect of the enteric soft capsule shell described herein, the film-forming polymer is gelatin.

Examples of enteric, acid-insoluble polymers are acrylic and methacrylate acid copolymers, cellulose acetate phthalate (CAP), cellulose acetate butyrate, hydroxypropylmethylcellulose phthalate (HPMCP), algenic acid salts such as sodium or potassium alginate, or shellac. Poly(methacylic acid-co-methyl methacrylate) anionic copolymers based on methacrylic acid and methyl methacrylate are particularly stable and are preferred in some embodiments. Poly(meth)acrylates (methacrylic acid copolymer), available under the trade name EUDRAGIT® (Evonik Industries AG, Essen, Germany), are provided as powder or aqueous dispersions. In some aspects, the methacrylic acid copolymer can be EUDRAGIT® L 30 D-55; EUDRAGIT® L 100-55; EUDRAGIT® L 100; EUDRAGIT® L 12.5; EUDRAGIT® S 100; EUDRAGIT® S 12.5; EUDRAGIT® FS 30 D; EUDRAGIT® E 100; EUDRAGIT® E 12.5; EUDRAGIT® E PO; EUDRAGIT® RL 100; EUDRAGIT® RL PO; EUDRAGIT® RL 30 D; EUDRAGIT® RL 12.5; EUDRAGIT® RS 100; EUDRAGIT® RS PO; EUDRAGIT® RS 30 D; EUDRAGIT® RS 12.5; EUDRAGIT® NE 30 D; EUDRAGIT® NE 40 D; EUDRAGIT® NM 30 D; or other poly(meth)acrylate polymers. In one aspect, the enteric polymer is EUDRAGIT® L 100, a methacrylic acid copolymer, Type A. Acid-insoluble polymer specifications are detailed in the United States Pharmacopoeia and in various monographs.

Plasticizers that are useful for creating enteric soft capsules as described herein comprise glycerol, sorbitol, polyethylene glycol, citric acid, citric acid esters, such as tri-ethyl citrate, or combinations thereof. The weight ratio between the film-forming polymer, the enteric acid-insoluble polymer, and plasticizer is adjusted so that the gel mass is flowable and not too viscous, and can be made into soft capsules using rotary die encapsulation methods.

In one embodiment, enteric soft capsule shell compositions can be made by dissolving the enteric acid-insoluble polymer in an aqueous solution of an alkali neutralizing agent such as ammonia, sodium hydroxide, potassium hydroxide, or liquid amines such as tri-ethanol amine or ethylene diamine. The amount of alkali is adjusted to give a final pH value of the gel mass less than or equal to about pH 9.0. In one embodiment, the final pH does not exceed 8.5. The volatile alkali neutralizing agent, ammonia is preferred. The film-forming polymer can then be combined with the plasticizer and solvent and then blended with the acid-insoluble gel to make a final homogeneous mix in a heat-controlled vessel and can be degassed by using vacuum. The fugitive ammonia evaporates during degassing. Using the foregoing process, the alkali concentrations do not require an additional step such as heating or neutralizing with acid in order to neutralize the gel mass.

In another embodiment described herein, the enteric soft capsule shell can be made by using an aqueous dispersion of the acid-insoluble polymer by adding alkaline materials such as ammonium, sodium, or potassium hydroxides, other alkalis, or a combination thereof that will cause the enteric acid-insoluble polymer to dissolve. The plasticizer-wetted, film-forming polymer can then be mixed with the solution of the acid-insoluble polymer. In one embodiment, enteric acid-insoluble polymers in the form of salts of the above-mentioned bases or alkalis can be dissolved directly in water and mixed with the plasticizer-wetted, film-forming polymer.

In one embodiment, the enteric soft capsule shell has the composition of Table 4, including all possible iterations of the specified ranges that provide 100% for the total weight percentage, including or excluding the optional, excipients, opacifiers, colorants, and flavorings.

TABLE 4
Exemplary Enteric Soft Capsule Shell Composition

| | | Composition |
|---|---|---|
| Component | Exemplary Component | Range (%) |
| Film-forming polymer | Gelatin | 20-36 |
| Enteric, acid insoluble polymer | Methacrylic Acid | 8-20 |
| | Copolymer | |
| Plasticizer | Glycerol, Triethyl citrate | 15-22 |
| Alkali neutralizing agents | NH₄OH (30%), NaOH | 1-5 |
| Solvent | Water | 20-40 |
| Opacifier | Titanium Dioxide | 1-7.5 |
| Colorant (optional) | Various | 0.05-1 |
| Flavoring (optional) | Various | 0.05-2 |
| Excipients (optional) | Various | 1-5 |

In one embodiment, the enteric soft capsule shell comprises a composition of about 30% film forming polymer; about 10% enteric, acid insoluble polymer; about 20% plasticizer; about 1% alkali neutralizing agent; and about 37% solvent.

In one embodiment, the weight percentage range of total polymer content (i.e., film forming polymer and enteric acid-insoluble polymer) of the enteric soft capsule described herein comprises from about 30% to about 45%, including all integers within the specified range. In one aspect, the total polymer weight percentage is about 40%. In another aspect, the total polymer weight percentage is about 42%. In another aspect, the total polymer weight percentage is about 45%. In another aspect, the total polymer weight percentage is about 38%.

In one embodiment, the weight percentage range of total plasticizer comprises from about 15% to about 22%, including all iterations of integers with the specified range. In one aspect, the total plasticizer weight percentage is about 19%. In another aspect, the total plasticizer weight percentage is about 17.7%. In another aspect, the total plasticizer weight percentage is about 18.9%. In another aspect, the total plasticizer weight percentage is about 19.3%.

In one embodiment, the alkali neutralizing-agent is ammonia (ammonium hydroxide; 30% w/v) that is added to comprise a weight percentage from about 1% to about 5% of the total enteric soft capsule composition. In one aspect, 30% w/v ammonia is added to comprise a weight percentage of about 2%. In another aspect, 30% w/v ammonia is added to a weight percentage of about 1.7%. In one aspect, ammonia is added to provide a final pH of about 9 in the enteric soft capsule composition. In another aspect, ammonia is added to provide a final pH of about 8.5 in the enteric soft capsule composition. In another aspect, after the capsules are filled and dried, the ammonia concentration is substantially reduced, owing to the fugitive nature of the volatile alkali. In some aspects, practically all of the ammonia is evaporated except for ammonium ions comprising salts with other moieties in the composition.

In one embodiment, the weight ratio range of film forming polymer to enteric acid insoluble polymer (film forming: enteric) comprises from about 25:75 (≈0.33) to about 40:60 (≈0.67) (i.e., ≈0.33-0.67), including all ratios within the specified range. In one aspect, the ratio of film forming polymer to enteric acid insoluble polymer is about 30:70 (≈0.43). In another aspect, the ratio of film forming polymer to enteric acid insoluble polymer is about 28:72 (≈0.38).

In one embodiment, the weight ratio of total plasticizer to film forming polymer comprises about 20:40 to 21:30 (i.e., ≈0.5-0.7), including all ratios within the specified range. In one aspect, the weight ratio of total plasticizer to film forming polymer is about 20:40 (≈4.5). In another aspect, the weight ratio of total plasticizer to film forming polymer is about 21:30 (≈4.7). In another aspect, the weight ratio of total plasticizer to film forming polymer is about 19:29 (≈0.65). In another aspect, the weight ratio of total plasticizer to film forming polymer is about 19.3:29.2 (≈0.66).

In one embodiment, the weight ratio of total plasticizer to enteric acid insoluble polymer is about 1:1 to about 2:1 (≈1-2), including all ratios within the specified range. In one aspect, the weight ratio of total plasticizer to enteric acid insoluble polymer is about 11:10 (≈1.1). In another aspect, the weight ratio of total plasticizer to enteric acid insoluble polymer is about 14:10 (≈1.4). In another aspect, the weight ratio of total plasticizer to enteric acid insoluble polymer is about 17:10 (≈1.7). In another aspect, the weight ratio of total plasticizer to enteric acid insoluble polymer is about 20:10 (≈2). In another aspect, the weight ratio of total plasticizer to enteric acid insoluble polymer is about 19.3:11.2 (≈21.73).

In one embodiment, the weight ratio range of total plasticizer to total polymer (film forming and enteric acid insoluble polymer) comprises from about 18:45 to about 20:40 (i.e., ≈0.40-0.5), including all ratios within the specified range. In one aspect, the weight ratio range of total plasticizer to total polymer is about 18:45 (≈0.40). In another aspect, the weight ratio range of total plasticizer to total polymer is about 19:40 (≈0.475). In another aspect, the weight ratio range of total plasticizer to total polymer is about 20:40 (≈0.5). In another aspect, the weight ratio range of total plasticizer to total polymer is about 19.3:40.4 (≈0.477).

In one embodiment, the solvent comprises from about 20% to about 40% of the enteric soft capsule composition, including all integers within the specified range. In one embodiment, the solvent is water. The quantity of water in the composition varies depending on the quantities of the other ingredients. For example, the quantity of opacifier, colorant, flavoring, or other excipients can change the percentage of water present in the composition. In one embodiment, the weight percentage of water is as much as suffices to bring the total weight percentage to 100% (i.e., quantum sufficiat; q.s.). In another embodiment, the water comprises about 20%, about 25%, about 30%, about 35%, or about 40% of the enteric soft capsule composition. In another embodiment, water comprises from about 35% to about 40% of the enteric soft capsule composition. In one embodiment, water comprises about 37% of the composition.

In one embodiment, the final moisture (water) content of the enteric soft capsule comprises from about 8% to about 15%, including all integers within the specified range. In another embodiment, the moisture content comprises from about 8% to about 12%, including all integers within the specified range. In one aspect, the final moisture content is about 8%. In one aspect, the final moisture content is about 9%. In one aspect, the final moisture content is about 10%. In one aspect, the final moisture content is about 11%. In another aspect, the final moisture content is about 12%.

In one embodiment, the enteric soft capsule shell has the exemplary composition shown in Table 5.

TABLE 5
Exemplary Enteric Soft Capsule Shell Composition

| | Percent |
|---|---|
| Component | weight (%) |
| Gelatin | 29.2 |
| Methacrylic Acid Copolymer (EUDRAGIT ® L 100) | 11.2 |
| Glycerol | 18.0 |
| Triethyl citrate | 1.3 |
| Ammonium hydroxide | 1.7 |
| Titanium dioxide | 1.5 |
| Water | 37.1 |
| TOTAL | 100 |
| Final pH | 8.5-9.0 |
| Total polymer % weight (gelatin + enteric) | 40.4 |
| Gelatin % weight of total polymer (gelatin + enteric) | 72.4 |
| Enteric % weight of total polymer (gelatin + enteric) | 27.6 |
| Ratio of Enteric to Gelatin | 11.2:29.2 (0.38) |
| Total plasticizer % weight (glycerol + triethyl citrate) | 19.3% |
| Ratio of total plasticizer to total polymer | 19.3:40.4 (0.48) |
| Ratio total plasticizer to gelatin | 19.3:29.2 (0.66) |
| Ratio total plasticizer to enteric | 19.3:11.2 (1.73) |
| Water content in dried enteric soft capsule: | 8-15 |

In one embodiment, the enteric soft capsule shell comprises about 30% gelatin; about 10% poly(methyl)acrylate copolymer; about 18% glycerol; about 1% triethyl citrate; about 1.7% ammonia; about 37% water; and about 1.5% titanium dioxide.

One embodiment described herein provides an enteric acid-insoluble polymer dispersed within the film-forming polymer gel mass that provides the total soft gel composition with enteric acid-insoluble properties, at relatively low concentrations of the enteric acid-insoluble polymer (e.g., from about 8% to about 20% of the total wet gel mass composition) and without the need of excessive amounts of alkali, thus avoiding denaturation or degradation of the film-forming polymer that can weaken the integrity of the enteric soft capsule shell.

Films of the enteric soft capsule shell do not dissolve or disintegrate in acids, such as 0.1 N hydrochloric acid or simulated gastric fluid (ca. pH 1.2), despite the fact that the majority of the shell ingredients (i.e., greater than 50%) normally dissolve in, or are miscible with, acids. Enteric soft capsules made using the compositions described herein remain intact in hydrochloric acid or simulated gastric fluid for at least two hours. The capsules readily release the contents upon shifting the pH of the solution to ca. 6.8, such as that of simulated intestinal fluid.

In another embodiment, the final enteric capsule composition provides films of increased strength without substantially compromising film elasticity. Moreover, films made from the enteric soft capsule compositions as described herein can be sealed at normal temperature range typically used for making traditional soft gel capsules. In one aspect, enteric soft capsules are made using a rotary die apparatus as described in U.S. Pat. Nos. 5,459,983; 5,146,730; and 6,482,516, each of which are incorporated by reference herein for such teachings.

Another embodiment described herein includes a process of manufacturing enteric soft capsules comprising the pharmaceutical composition as described herein. The process includes preparing a gel mass composition comprising a film-forming, water-soluble polymer and an enteric acid-insoluble polymer and mixing with appropriate plasticizers and solvent; casting the gel mass into films or ribbons using heat-controlled drums or surfaces; and manufacturing a soft capsule comprising a matrix fill using rotary die technology. The thickness of the films or ribbons that form the enteric capsule shell comprises from about 0.010 inches (≈0.254 mm) to about 0.050 inches (≈1.27 mm), including all integers within the specified range. The shell thickness can be about 0.010 inch (≈0.254 mm), about 0.015 inch (≈0.381 mm), about 0.02 in (≈0.508 mm), about 0.03 in (≈0.762 mm), about 0.04 in (≈1.02 mm), or about 0.05 in (≈1.27 mm). In one embodiment, the thickness is about 0.02 inches (≈0.508 mm) to about 0.040 inches (≈1.02 mm). In one embodiment, the shell thickness is about 0.028 inches (≈0.711 mm). In another embodiment, the shell thickness is about 0.033 inches (≈0.838 mm). In another embodiment, the shell thickness is about 0.038 inches (≈0.965 mm).

The enteric soft capsules described herein can contain a matrix fill that is liquid, semi-solid, or solid. Capsules prepared as described herein can contain a hydrophobic solution or suspension, such as vegetable oils or shortening, or waxes, or combinations thereof. The matrix fill can be formulated to prevent interaction with the capsule shell components and release the pharmaceutical composition at a specified rate.

In another embodiment, the pharmaceutical composition described herein provides a dosage of an active pharmaceutical ingredient described herein. The dosage form can be administered, for example, to a subject, or a subject in need thereof. In one aspect, the subject may be a mammal, or a mammal in need thereof. In one aspect, the dosage form can be administered, for example, to a human or a human in need thereof. In one aspect, the human subject or a human subject in need thereof is a medical patient.

In one embodiment, the oral pharmaceutical composition described herein, comprises an active pharmaceutical ingredient of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, or even more.

In another embodiment, the oral pharmaceutical composition described herein, comprises an active pharmaceutical ingredient in the range of about 5 mg to about 75 mg, about 10 mg to about 75 mg, about 15 mg to about 75 mg, about 20 mg to about 75 mg, about 25 mg to about 75 mg, about 30 mg to about 75 mg, about 35 mg to about 75 mg, about 40 mg to about 75 mg, about 45 mg to about 75 mg, about 50 mg to about 75 mg, about 55 mg to about 75 mg, about 60 mg to about 75 mg, about 65 mg to about 75 mg, or about 70 mg to about 75 mg.

In another embodiment, the oral pharmaceutical composition described herein, comprises an active pharmaceutical ingredient in the range of about 10 mg to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 110 mg to about 150 mg, about 120 mg to about 150 mg, about 130 mg to about 150 mg, or about 140 mg to about 150 mg.

In one embodiment, the active pharmaceutical ingredient is dabigatran, or a salt, ether, ester, variant, or derivative thereof. In one embodiment, the active pharmaceutical ingredient is dabigatran etexilate or a pharmaceutically acceptable salt form. Pharmaceutically acceptable salts forms are those formed by contacting dabigatran etexilate free base with a suitable acid in a suitable solvent under suitable conditions that will react to form dabigatran etexilate acid addition salt. Suitable acids include hydrochloric acid, camphorsulfonic acid, hydrobromic acid, sulfuric acid, methanesulfonic acid, formic acid, acetic acid, oxalic acid, succinic acid, tartaric acid, mandelic acid, malic acid, salicylic acid, fumaric acid, lactic acid, citric acid, glutamic acid, and/or aspartic acid. In one aspect, the active pharmaceutical ingredient is the mesylate salt of dabigatran etexilate (e.g., dabigatran etexilate mesylate; DEM).

In one embodiment, dabigatran is in the form of a double prodrug (e.g., dabigatran etexilate mesylate) that is rapidly absorbed. Without being bound by any theory, dabigatran etexilate mesylate is converted to its active form (e.g., dabigatran) by non-specific plasma and hepatic esterases into its active moiety that cleave two polar ester groups. The active form of dabigatran and its acyl glucuronides are competitive, direct thrombin inhibitors. Because thrombin (serine protease) enables the conversion of fibrinogen into fibrin during the coagulation cascade, its inhibition prevents the development of a thrombus. Both free and clot-bound thrombin and thrombin-induced platelet aggregation are inhibited by the active moieties.

In another embodiment described herein, is a method for orally administering a dosage form of dabigatran for the treatment, amelioration, prophylaxis, or reducing the onset of or symptoms of a coagulation disorder.

In one aspect, the dose of dabigatran comprised by the pharmaceutical composition is about 20 mg. In another aspect, the dose of dabigatran comprised by the pharmaceutical composition is about 25 mg. In another aspect, the dose of dabigatran comprised by the pharmaceutical composition is about 50 mg. In another aspect, the dose of dabigatran comprised by the pharmaceutical composition is about 75 mg. In another aspect, the dose of dabigatran comprised by the pharmaceutical composition is about 100 mg. In another aspect, the dose of dabigatran comprised by the pharmaceutical composition is about 110 mg. In another aspect, the dose of dabigatran comprised by the pharmaceutical composition is about 150 mg. In another aspect, the dose of dabigatran comprised by the pharmaceutical composition is about 200 mg. In another aspect, the dose of dabigatran comprised by the pharmaceutical composition is about 250 mg. In another aspect, the dose of dabigatran comprised by the pharmaceutical composition is about 300 mg. In another aspect, the dose of dabigatran comprised by the pharmaceutical composition is about 350 mg. In another aspect, the dose of dabigatran comprised by the pharmaceutical composition is about 400 mg. In another aspect, the dose of dabigatran comprised by the pharmaceutical composition is about 450 mg. In another aspect, the dose of dabigatran comprised by the pharmaceutical composition is about 500 mg.

In another embodiment, the total dosage of dabigatran administered in a 24-hour period comprises from about 20 mg to about 600 mg per 24-hour period. In one aspect, the total dosage of dabigatran administered in a 24-hour period is about 50 mg to about 250 mg per 24-hour period. In another aspect, the total dosage of dabigatran administered in a 24-hour period is about 300 mg per 24-hour period.

In another embodiment, the dabigatran comprised by the pharmaceutical composition described herein is in a particulate form. In one aspect, dabigatran can be micronized or non-micronized. In another aspect, micronized dabigatran has a particle size ranging from about 0.5 μm to about 20 μm. In another aspect, micronized dabigatran has a particle size of about 1 μm. In another aspect, micronized dabigatran has a particle size of about 4 μm. In another aspect, micronized dabigatran has a particle size of about 14 μm. In another aspect, non-micronized dabigatran has a particle size ranging from about 4 μm to about 75 μm. In another aspect, non-micronized dabigatran has a particle size of about 5 μm. In another aspect, non-micronized dabigatran has a particle size of about 21 μm. In another aspect, non-micronized dabigatran has a particle size of about 50 μm.

In another embodiment, the dosage can contain a total amount of dabigatran effective for treatment, amelioration, prophylaxis, or reducing the onset of or symptoms of a coagulation disorder. In one aspect, the dosage can contain a total amount of dabigatran effective for treatment, amelioration, prophylaxis, or reducing the onset of or symptoms of stroke. In another aspect, the dosage can contain a total amount of dabigatran effective for treatment, amelioration, prophylaxis, or reducing the onset of or symptoms of ischemic stroke. In another aspect, the dosage can contain a total amount of dabigatran effective for treatment, amelioration, prophylaxis, or reducing the onset of or symptoms of hemorrhagic stroke. In another aspect, the dosage can contain a total amount of dabigatran effective for treatment, amelioration, prophylaxis, or reducing the onset of or symptoms of thrombosis. In another aspect, the dosage can contain a total amount of dabigatran effective for treatment, amelioration, prophylaxis, or reducing the onset of or symptoms of pulmonary embolism. In another aspect, the dosage can contain a total amount of dabigatran effective for treatment, amelioration, prophylaxis, or reducing the onset of or symptoms of clotting in patients with non-valvular atrial fibrillation. In another aspect, the dosage can contain a total amount of dabigatran effective for treatment, amelioration, prophylaxis, or reducing the onset of or symptoms of systemic embolism in patients with non-valvular atrial fibrillation. In another aspect, the dosage can contain a total amount of dabigatran effective for treatment, amelioration, prophylaxis, or reducing the onset of or symptoms of deep vein thrombosis. In another aspect, the dosage can contain a total amount of dabigatran effective for treatment, amelioration, prophylaxis, or reducing the onset of or symptoms of myocardial infarction. In another aspect, the dosage can contain a total amount of dabigatran effective for treatment, amelioration, prophylaxis, or reducing the onset of or symptoms of antiphospholipid syndrome. In another aspect, the dosage can contain a total amount of dabigatran effective for treatment, amelioration, prophylaxis, or reducing the onset of or symptoms of embolism in patients with prosthetic heart valves.

In one embodiment, dabigatran is administered in patients previously treated with a parenteral anticoagulant for a time period of about 1 day to about 20 days. In another embodiment, dabigatran is administered in patients previously treated with a parenteral anticoagulant for a time period of about 5 days to about 10 days. Exemplary parenteral anticoagulants comprise: heparin (e.g., unfractionated heparin (UFH) and low molecular weight heparin (LMWH)), fondaparinux, danaparoid, recombinant hirudins (e.g., lepirudin and desirudin), bivalirudin, and argatroban or a mixture of parenteral anticoagulants thereof. In one aspect, the dosage of dabigatran can contain a total amount of dabigatran effective for treatment, amelioration, prophylaxis, or reducing the onset of or symptoms of deep vein thrombosis in patients previously treated with a parenteral anticoagulant. In another aspect, the dosage of dabigatran can contain a total amount of dabigatran effective for treatment, amelioration, prophylaxis, or reducing the onset of or symptoms of pulmonary embolism in patients previously treated with a parenteral anticoagulant.

The dosage form can be administered, for example, 1×, 2×, 3×, 4×, 5×, or 6× per day. One or more dosage form can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, or even longer. One or more dosage forms can be administered until the patient, subject, mammal, mammal in need thereof, human, or human in need thereof, does not require treatment, prophylaxis, or amelioration of any disease or condition such as, for example, a coagulation disorder.

In another embodiment, the dosage forms as described herein can be administered with or without food. In one aspect, the dosage forms as described herein can be administered with food. In another aspect, the dosage forms as described herein can be administered without food.

In another embodiment, the total dosage of dabigatran administered in a 24-hour period is about 300 mg administered twice daily. In one aspect, the total dosage of dabigatran administered in a 24-hour period is about 300 mg administered twice daily effective for the treatment, amelioration, prophylaxis, or reducing the onset of or symptoms of stroke in non-valvular atrial fibrillation. In another aspect, the total dosage of dabigatran administered in a 24-hour period is about 300 mg administered twice daily effective for the treatment, amelioration, prophylaxis, or reducing the onset of or symptoms of systemic embolism in non-valvular atrial fibrillation. In another aspect, the total dosage of dabigatran administered in a 24-hour period is about 300 mg administered twice daily effective for the treatment, amelioration, prophylaxis, or reducing the onset of or symptoms of deep vein thrombosis. In another aspect, the total dosage of dabigatran administered in a 24-hour period is about 300 mg administered twice daily effective for the treatment, amelioration, prophylaxis, or reducing the onset of or symptoms of pulmonary embolism.

In another embodiment, the pharmaceutical composition described herein comprising dabigatran is administered to a human patient in need thereof in a single dosage form of about 10 mg to about 400 mg. In one aspect, the pharmacokinetic profile of dabigatran is dose proportional (e.g., the individual pharmacokinetic parameters increase in proportion with increasing dosage amounts and frequency of administered dabigatran). In one aspect, the absolute bioavailability of dabigatran is from about 1% to about 20%. In another aspect, the absolute bioavailability of dabigatran is from about 3% to about 10%. In another aspect, the absolute bioavailability of dabigatran is about 3%, about 4%, about 5%, about 6%, about 7%, about 8%, about 9%, or even about 10%. In another aspect, the Cₘₐₓ of dabigatran ranges from about 5 ng/mL to about 500 ng/mL. In another aspect, the Tₘₐₓ occurs at about 0.5 hours to about 3 hours post-administration. In another aspect, the half-life of dabigatran is from about 8 hours to about 24 hours. In another aspect, the AUc_{0→∝} is from about 200 ng·h/mL to about 5500 ng·h/mL. In another aspect, the above pharmacokinetic parameters of the pharmaceutical composition described herein comprising dabigatran may vary with patient population and the administration of other drugs to the patient or patient population.

In another embodiment, the matrix fills comprised by the pharmaceutical composition described herein prevent the degradation of dabigatran etexilate mesylate (e.g., from hydrolytic cleavage, enantiomeric conversion, photodegradation or contamination). In one aspect, the percent of dabigatran etexilate mesylate degradation products or other impurities in the matrix fill is less than about 2%. In another aspect, the percent of dabigatran etexilate mesylate degradation products or other impurities comprise: ethyl 3-(2-(((4-carbamimidoylphenyl)amino)methyl)-1-methyl-N(pyridin-2-yl)-1H-benzo[d]imidazole-5-carboxamido)propanoate methanesulfonate (DBG-2A); 3-(2-(((4-(N((hexyloxy)carbonyl)carbamimidoyl)phenyl)amino)methyl)-1-methyl-N-(pyridin-2-yl)-1H-benzo[d]imidazole-5-carboxamido)propanoic acid methanesulfonate (DBG-3A Acid); ethyl 2-(((4-(N((hexyloxy)carbonyl)carbamimidoyl)phenyl)amino)methyl)-1-methyl-1Hbenzo[d]imidazole-5-carboxylate methanesulfonate (DBG-3A Ester); Ethyl 3 -(2-(((4-(((hexyloxy)carbonyl)carbamoyl)phenyl)amino)methyl)-1-methyl-N-(pyridin-2-yl)-1Hbenzo[d]imidazole-5-carboxamido)propanoate methanesulfonate (DBG-3A Amide), Ethyl 3-(3-(((hexyloxy)carbonyl)amino)-4-(2-((4-(N′-((hexyloxy)carbonyl)carbamimidoyl)phenyl)amino)-N-methylacetamido)-N-(pyridin-2-yl)benzamido)propanoate methanesulfonate (DBG-3A Dihexyl); Hexyl((4-(((5-((3-amino-3-oxopropyl)(pyridin-2-yl)carbamoyl)-1-methyl-1Hbenzo[d]imidazol-2-yl)methyl)amino)phenyl)(imino)methyl)carbamate methanesulfonate (DBG-3A Hexyl Amide); 2-(((4-(N-((hexyloxy)carbonyl)carbamimidoyl)phenyl)amino)methyl)-1-methyl-1H-benzo[d]imidazole-5-carboxylic acid methanesulfonate (DBG-3A Des Amino Pyridine); ethyl 3 -(2-(((4-cyanophenyl)amino)methyl)-1-methyl-N-(pyridin-2-yl)-1H-benzo[d]imidazole-5-carboxamido)propanoate methanesulfonate (CBA); Methyl 3-(2-(((4-(N((hexyloxy)carbonyl)carbamimidoyl)phenyl)amino)methyl)-1-methyl-N-(pyridin-2-yl)-1H-benzo[d]imidazole-5-carboxamido)propanoate methanesulfonate (DBG-3A Methoxy), or diaryltriazinone, or a combination thereof. Impurities can be detected by using methodologies known by persons skilled in the art, e.g., high performance liquid chromatography (HPLC), mass spectrometry, or nuclear magnetic resonance (NMR), among other methods.

In another embodiment, the pharmaceutical composition described herein is contained and dispensed from a tamper evident packaging. The term “tamper evident” or “tamper resistant” refers to a packaging of any kind that readily displays or allows an individual to observe any physical interference or manipulation of said packaging. The tamper evident packaging provides reasonable evidence to consumers that tampering has occurred. The tamper evident packaging additionally contains appropriate labelling statements describing the features and evidences of the tamper evident packaging. In one aspect, the tamper evident packaging comprises: bottles, film wrappers, blister or strip packs, bubble packs, heat shrink bands or wrappers, foil, paper, or plastic pouches, container mouth inner seals, tape seals, breakable caps, sealed metal tubes or plastic heat-sealed tubes, sealed cartons, aerosol containers, cans including metal and composite materials, or any combination thereof. The packaging may also contain appropriate instructions for prescribing, instructions for use, warnings, or other appropriate information.

In another embodiment, the pharmaceutical composition comprising the active pharmaceutical ingredient described herein does not undergo the degradation processes as described herein following the opening of the tamper evident packaging described herein comprising said pharmaceutical composition. In one aspect, the active pharmaceutical ingredient is stable for a time period of about 60 days to about 360 days following the opening of the tamper evident packaging described herein comprising said pharmaceutical composition described. In another aspect, the active pharmaceutical ingredient is stable for a time period of at least about 90 days following the opening of the tamper evident packaging described herein comprising said pharmaceutical composition. In another aspect, the active pharmaceutical ingredient is stable for a time period of at least about 120 days following the opening of the tamper evident packaging described herein comprising said pharmaceutical composition. In another aspect, the active pharmaceutical ingredient is stable for a time period of at least about 180 days following the opening of the tamper evident packaging described herein comprising said pharmaceutical composition. In another aspect, the active pharmaceutical ingredient is stable for a time period of at least about 200 days, at least about 225 days, at least about 250 days, at least about 275 days, at least about 300 days, at least about 325 days, or even at least about 360 days following the opening of the tamper evident packaging described herein comprising said pharmaceutical composition.

It will be readily apparent to one of ordinary skill in the relevant arts that suitable modifications and adaptations to the compositions, methods, and applications described herein can be made without departing from the scope of any embodiments or aspects thereof The compositions and methods provided are exemplary and are not intended to limit the scope of any of the specified embodiments. All of the various embodiments, aspects, and options disclosed herein can be combined in any and all variations or iterations. The scope of the compositions, formulations, methods, and processes described herein include all actual or potential combinations of embodiments, aspects, options, examples, and preferences herein described. The ratios of the mass of any component of any of the formulations disclosed herein to the mass of any other component in the formulation or to the total mass of the other components in the formulation are hereby disclosed as if they were expressly disclosed. All patents and publications cited herein are incorporated by reference herein for the specific teachings thereof.

### EXAMPLES

### Example 1

Examples of matrix fill compositions highlighting the plurality of lipid or lipophilic vehicles useful for the pharmaceutical composition described herein are shown below in Table 6. The composition of each component is set forth by weight percentage of the total weight of the matrix fill.

TABLE 6
Exemplary Matrix Fill Compositions

| | Percent Weight (%) | | | | | |
|---|---|---|---|---|---|---|
| Ingredient | EX 1 | EX 2 | EX 3 | EX 4 | EX 5 | EX 6 |
| Soybean oil | 68 | 90 | 0 | 0 | 0 | 0 |
| Fish oil | 0 | 0 | 75 | 0 | 0 | 0 |
| Miglyol ® | 0 | 0 | 0 | 65 | 0 | 0 |
| Paceol ™ | 0 | 0 | 0 | 0 | 62 | 0 |
| Capryol ™ | 0 | 0 | 0 | 0 | 0 | 65 |
| Non-ionic | 12 | 0 | 0 | 15 | 10 | 10 |
| surfactant | | | | | | |
| Antioxidant | 0.01 | 0.01 | 0.01 | 0.01 | 0.01 | 0.01 |
| Absorption | 0 | 0 | 5 | 5 | 5 | 5 |
| enhancer | | | | | | |
| Active | 20 | 10 | 20 | 15 | 23 | 20 |
| pharmaceutical | | | | | | |
| ingredient | | | | | | |
| TOTAL | 100 | 100 | 100 | 100 | 100 | 100 |

### Example 2

Examples of matrix fill compositions highlighting the plurality of non-ionic surfactants useful for the pharmaceutical composition described herein are shown below in Table 7. The composition of each component is set forth by weight percentage of the total weight of the matrix fill.

TABLE 7
Exemplary Matrix Fill Compositions

| | Percent Weight (%) | | | | | |
|---|---|---|---|---|---|---|
| Ingredient | EX 1 | EX 2 | EX 3 | EX 4 | EX 5 | EX 6 |
| Lipid or lipophilic | 0 | 0 | 74 | 68 | 50 | 0 |
| vehicle | | | | | | |
| Lauroglycol ™ | 60 | 0 | 0 | 0 | 16 | 65 |
| Labrasol ® | 0 | 70 | 0 | 0 | 0 | 0 |
| TWEEN ® | 10 | 0 | 10 | 0 | 10 | 0 |
| Span ® | 0 | 0 | 0 | 12.5 | 10 | 10 |
| Antioxidant | 0.01 | 2 | 0.5 | 0.01 | 0.05 | 0.01 |
| Water-binding | 5 | 0 | 3 | 0 | 4 | 5 |
| excipient | | | | | | |
| Absorption enhancer | 5 | 5 | 2.5 | 0 | 0 | 0 |
| Active | 20 | 23 | 10 | 20 | 10 | 20 |
| pharmaceutical | | | | | | |
| ingredient | | | | | | |
| TOTAL | 100 | 100 | 100 | 100 | 100 | 100 |

### Example 3

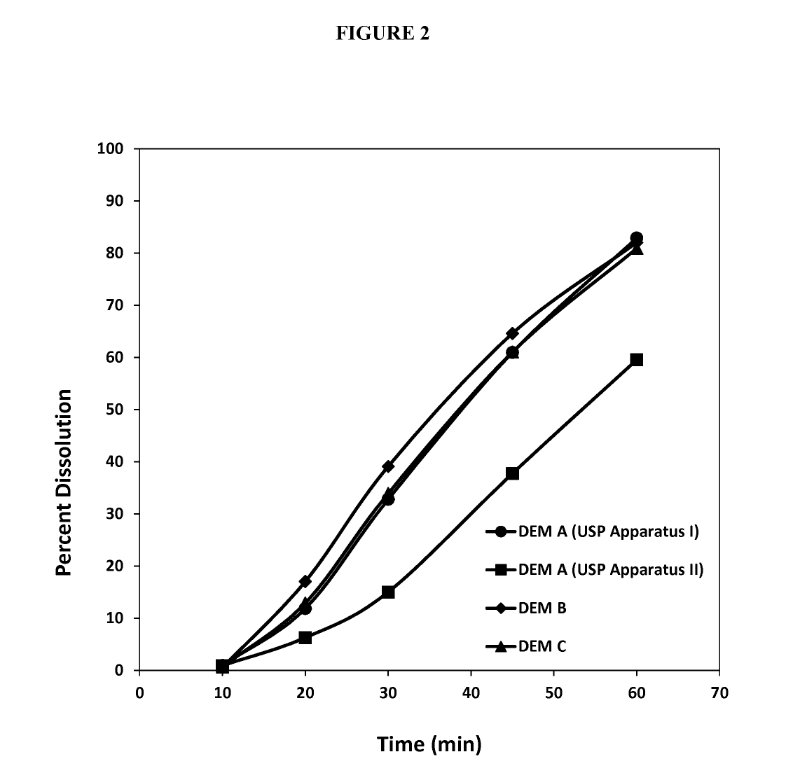

Gelatin soft capsules comprising a matrix fill as described herein were prepared using the composition shown in Table 8. The composition of each component is set forth by weight percentage of the total weight of the soft capsule shell or the matrix fill. The dissolution kinetics of the different pharmaceutical composition formulations shown in Table 8 were tested. These studies were conducted with either USP apparatus I or USP apparatus II in replicate vessels containing 900 mL of 2% cetrimonium bromide (CTAB) in 0.01 N HCl dissolution media. Samples were collected at 10 minutes, 20 minutes, 30 minutes, 45 minutes, and 60 minutes. The amount of dissolved dabigatran etexilate mesylate was determined by HPLC analysis and is shown in FIG. 2.

TABLE 8
Exemplary Soft Capsule Shell with Matrix Fill Formulation
Soft Capsule Shell Composition

| Ingredient | Percent Weight (%) |
|---|---|
| Gelatin | 43 |
| Glycerol | 20 |
| Titanium dioxide | 0.7 |
| Water | 36.2 |
| TOTAL | 100 |

Matrix Fill Composition

| | Percent Weight (%) | | |
|---|---|---|---|
| Ingredient | DEM A | DEM B | DEM C |
| Lauroglycol ™ 90 | 62 | 0 | 0 |
| Capryol ™ 90 | 0 | 62 | 0 |
| Labrasol ® | 0 | 0 | 72 |
| Vitamin E TPGS | 5 | 5 | 5 |
| TWEEN ® 80 | 10 | 10 | 0 |
| Dabigatran (DEM) | 23 | 23 | 23 |
| BHT | 0.01 | 0 | 0 |
| TOTAL | 100 | 100 | 100 |

### Example 4

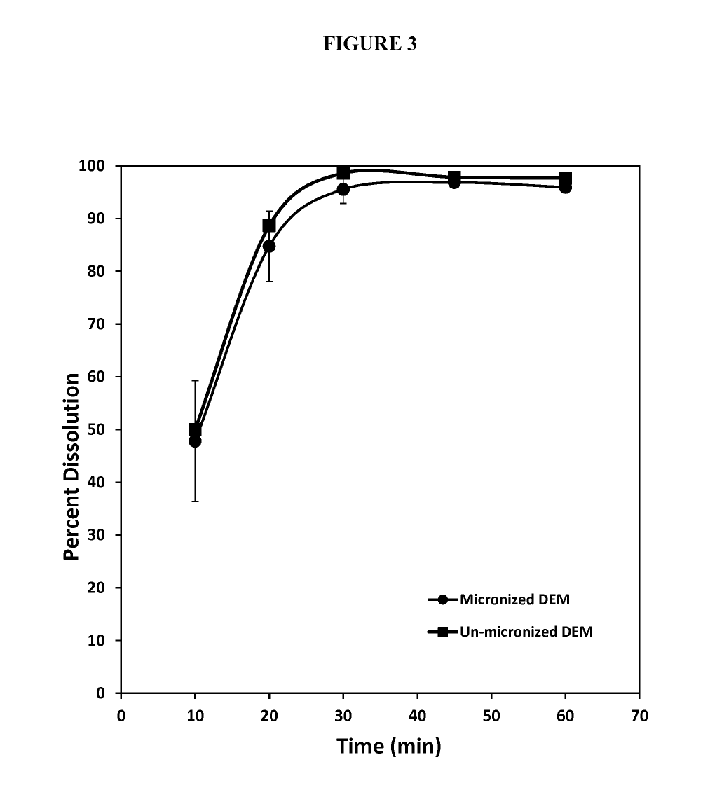

Examples of dabigatran etexilate mesylate particle size useful for the pharmaceutical composition described herein are shown in Table 9. The distribution of dabigatran etexilate mesylate particle sizes is set forth by the diameter distribution in either micronized or non-micronized samples. The dissolution of micronized and non-micronized dabigatran etexilate mesylate particles was studied using the matrix fill corresponding to DEM A as shown in Table 8 encapsulated in a hard gelatin capsule. The effects of dabigatran etexilate mesylate particle size on dabigatran etexilate mesylate dissolution are shown in FIG. 3.

TABLE 9
Exemplary Particle Sizes of Micronized and Non-micronized
Dabigatran (DEM)

| DEM Particle | D(v, 0.9) | D(v, 0.5) | D(v, 0.1) |
|---|---|---|---|
| Micronized | 10.5 μm | 4.2 μm | 1.5 μm |
| Non-micronized | 50.1 μm | 20.8 μm | 4.7 μm |

### Example 5

### Manufacturing Process

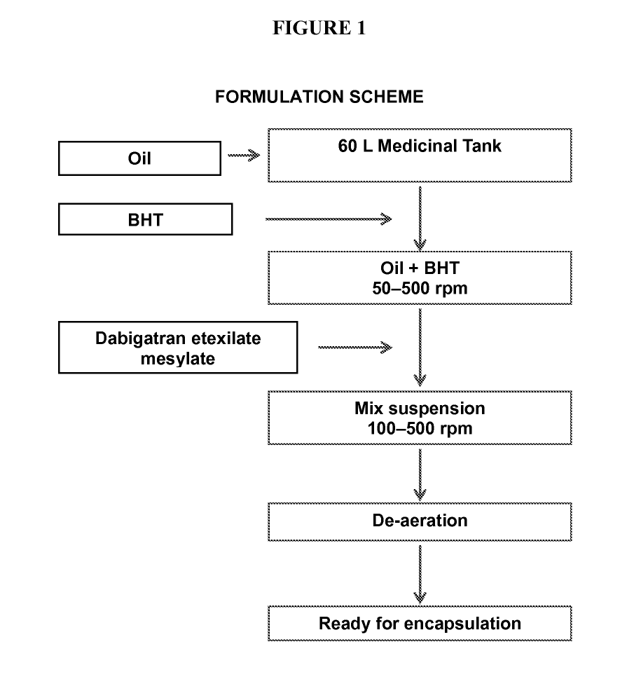

The manufacturing process of the immediate release matrix fill compositions as described herein is illustrated in FIG. 1. First, the lipid or lipophilic vehicle comprising one or more oils as described herein is added to a vessel of a suitable size. Next, one or more pharmaceutically acceptable excipients comprising BHT is added to said vessel and dissolved at about room temperature with an agitator rotating at about 50 RPM to about 500 RPM until a clear solution is formed. Following the formation of a clear solution step, the active pharmaceutical ingredient comprising dabigatran etexilate mesylate is added in an appropriate amount for the desired dosages as described herein and homogenized at about room temperature at about 100 RPM to about 500 RPM until a homogenous suspension is formed (e.g., suspension free of lumps). Following the mixing step and homogenization step, the mixture comprising dabigatran etexilate mesylate is de-aerated until substantially free of air (e.g., no air bubbles or other sign of air is observable). Following the de-aeration step, the matrix fill composition as described herein is mixed with an agitator rotating at about 50 RPM to about 200 RPM and is encapsulated in the soft capsule shells described herein using rotary die encapsulation technology.

### Example 6

Gelatin soft capsules comprising a matrix fill as described herein were prepared using the composition shown in Table 10.

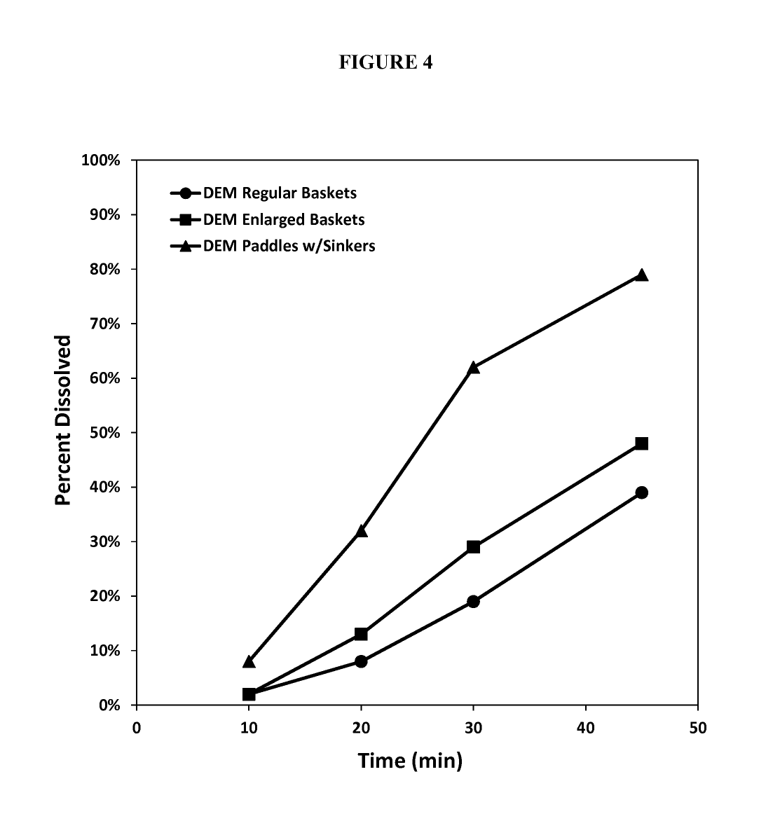

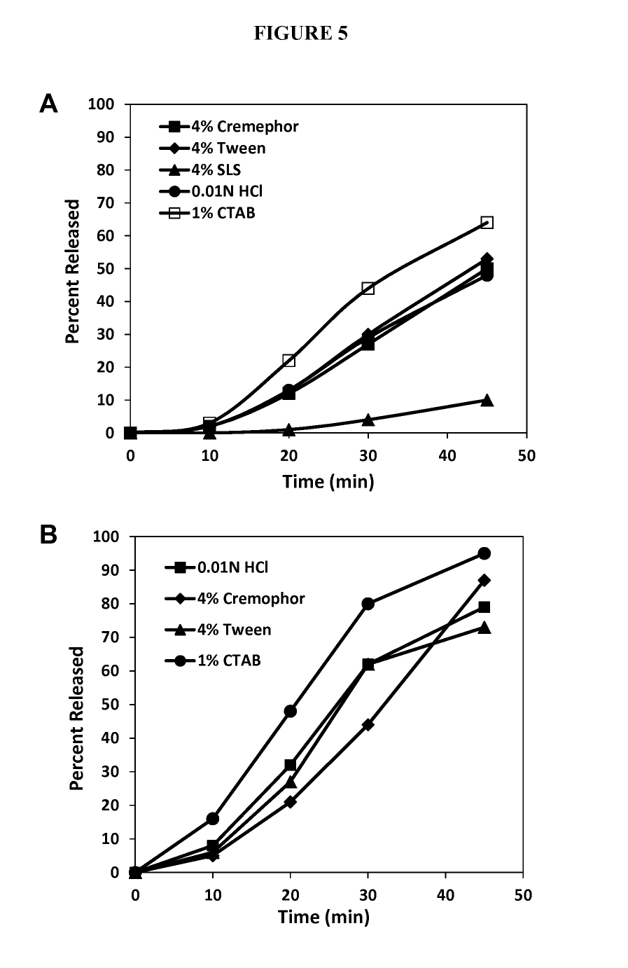

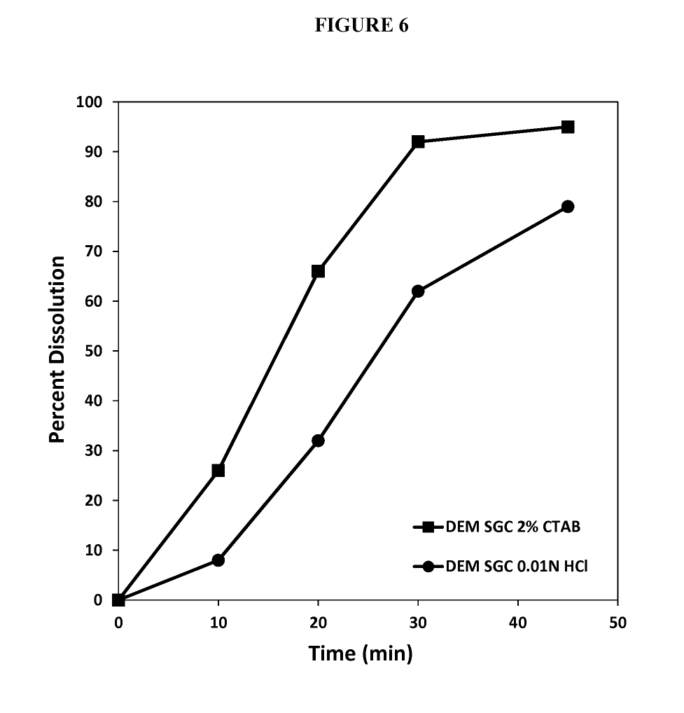

The dissolution conditions were further optimized using the pharmaceutical composition shown in Table 10. The following studies were conducted in vessels containing a dissolution media, wherein the capsules were dipped under agitating paddles (rotating at about 100 RPM); samples were collected at 10 minutes, 20 minutes, 30 minutes, and 45 minutes and were analyzed by HPLC analysis. The dissolution kinetics of dabigatran etexilate mesylate in 0.01 N HCl using regular baskets, enlarged baskets, or paddles with sinkers is shown in FIG. 4. The effects of different surfactants on the release kinetics of dabigatran etexilate mesylate are shown in panels A and B of FIG. 5. The effects of 2% cetrimonium bromide (CTAB) on the dissolution kinetics of dabigatran etexilate mesylate are shown in FIG. 6.

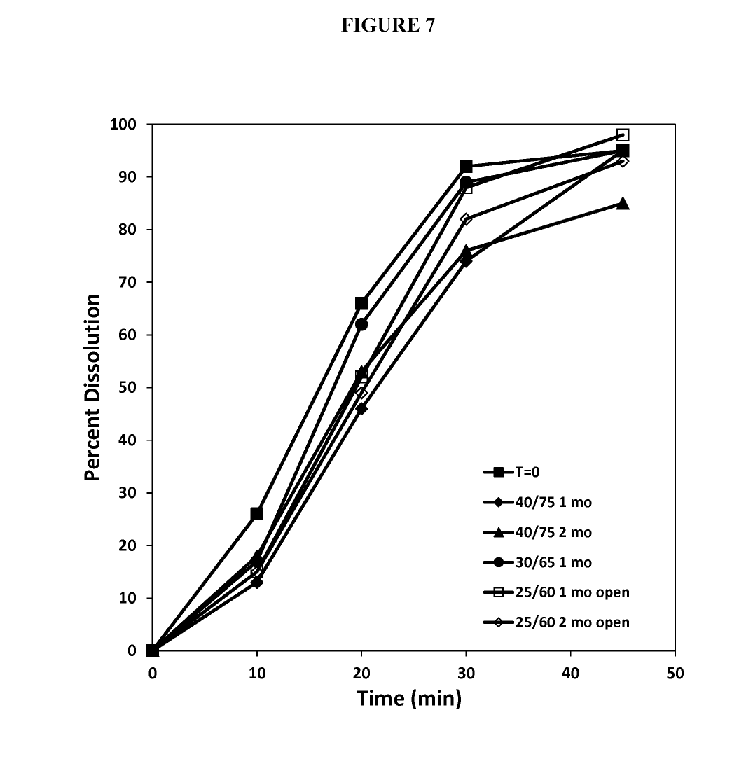

In addition, the effects of different storage conditions of the pharmaceutical composition shown in Table 10 were tested. Different samples were stored for 0 months, 1 month or 2 months at 40° C. at a relative humidity of 75% in a closed container, 1 month at 30° C. at a relative humidity of 65% in a closed container, and 1 month or 2 months at 25° C. at a relative humidity of 60% in an opened container. The effects of these different conditions were tested in a dissolution media containing 2% CTAB in 0.01 N HCl and are shown in FIG. 7.

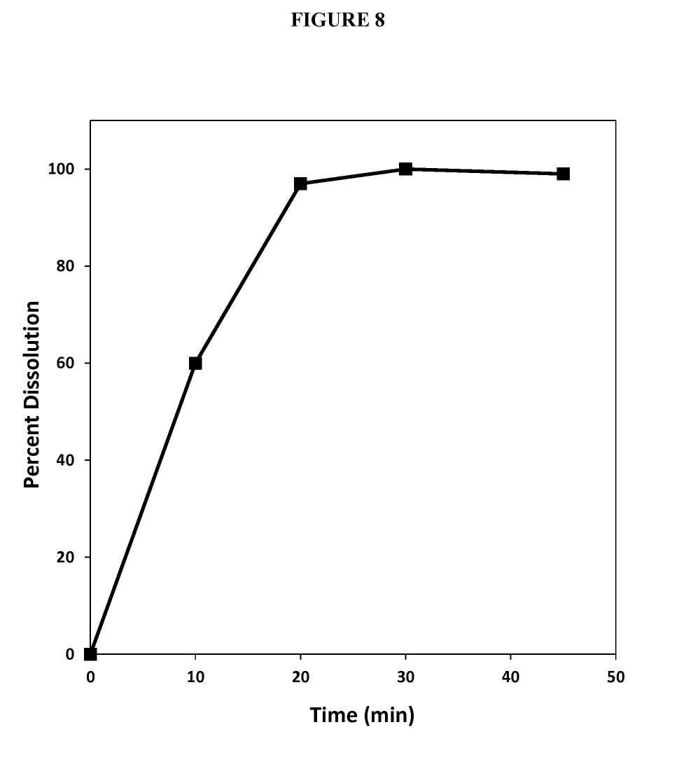

The pharmaceutical composition shown in Table 10 was further tested for its dissolution kinetics in the optimized dissolution media. These studies were conducted in replicate vessels containing 900 mL of 2% cetrimonium bromide (CTAB) in 0.01 N HCl dissolution media. The capsules were dipped 10 times per minute in the media under agitating paddles (rotating at about 100 RPM), and samples were collected at 10 minutes, 20 minutes, 30 minutes, and 45 minutes. The amount of dissolved dabigatran etexilate mesylate was determined by HPLC analysis and is shown in FIG. 8.

TABLE 10
Exemplary Soft Capsule Shell with Matrix Fill Formulation
Soft Capsule Shell Composition

| Ingredient | Percent Weight (%) |
|---|---|
| Gelatin | 43 |
| Glycerol | 20 |
| Titanium dioxide | 0.7 |
| Water | 36.2 |
| TOTAL | 100 |

Matrix Fill Composition

| Ingredient | Weight (mg/capsule) | Percent Weight (%) |
|---|---|---|
| Soybean oil | 727.4 | 80.8 |
| Dabigatran (DEM) | 172.5 | 19.2 |
| BHT | 0.1 | 0.01 |
| TOTAL | 900 | 100 |

### Example 7

Gelatin soft capsules comprising a matrix fill as described herein were prepared using the composition shown in Table 11.

TABLE 11
Exemplary Soft Capsule Shell with Matrix Fill Formulation
Soft Capsule Shell Composition

| Ingredient | Percent Weight (%) |
|---|---|
| Gelatin | 43 |
| Glycerol | 20 |
| Titanium dioxide | 0.7 |
| Water | 36.2 |
| TOTAL | 100% |

Matrix Fill Composition

| Ingredient | Weight (mg/capsule) | Percent Weight (%) |
|---|---|---|
| Miglyol ® 812 | 614.9 | 68 |
| TWEEN ® 80 | 112.5 | 12.5 |
| Dabigatran (DEM) | 172.5 | 19.2 |
| BHT | 0.1 | 0.01 |
| TOTAL | 900 | 100 |

### Example 8

Gelatin soft capsules comprising a matrix fill as described herein were prepared using the composition shown in Table 12.

TABLE 12
Exemplary Soft Capsule Shell with Matrix Fill Formulation
Soft Capsule Shell Composition

| Ingredient | Percent Weight (%) |
|---|---|
| Gelatin | 43 |
| Glycerol | 20 |
| Titanium dioxide | 0.7 |
| Water | 36.2 |
| TOTAL | 100 |

Matrix Fill Composition

| Ingredient | Weight (mg/capsule) | Percent Weight (%) |
|---|---|---|
| Soybean oil | 614.9 | 68 |
| TWEEN ® 80 | 112.5 | 12.5 |
| Dabigatran (DEM) | 172.5 | 19.2 |
| BHT | 0.1 | 0.01 |
| TOTAL | 900 | 100 |

### Example 9

The pharmaceutical composition described herein comprising a soft gelatin capsule shell and a matrix fill as described herein were prepared in a batch using the composition shown in Table 10 and the stability of dabigatran etexilate mesylate comprised by the matrix fill was tested and compared to commercially available capsules as shown in Table 13. The soft gelatin capsules described herein were stored in sealed bottles for a period of one or two months at either 30° C. at 65% relative humidity (Test A) or at 40° C. at 75% relative humidity (Test B).

TABLE 13
Stability and Percent Purity (%) of the Pharmaceutical Composition Comprising
Dabigatran (DEM)

| | Commercial | EX Batch | Test A | Test B | Test B |
|---|---|---|---|---|---|
| Impurity | 0 Months | 0 Months | 1 Month | 1 Month | 2 Months |
| DBG-2A | <0.05 | <0.05 | <0.05 | 0.05 | <0.05 |
| DBG-3A Acid | 0.05 | <0.05 | <0.05 | <0.05 | 0.05 |
| DBG-3A Ester | ND | <0.05 | <0.05 | <0.05 | <0.05 |
| DBG-3A Amide | 0.12 | 0.07 | 0.12 | 0.15 | 0.16 |
| DBG-3A Dihexyl | <0.05 | <0.05 | <0.05 | <0.05 | <0.05 |
| DBG-3A Hexyl amide | <0.05 | ND | <0.05 | <0.05% | <0.05 |
| DBG-3A Des | ND | ND | ND | ND | ND |
| Amino Pyridine | | | | | |
| CBA | ND | ND | <0.05 | ND | ND |
| DBG-3A Methoxy | <0.05 | ND | ND | <0.05 | <0.05 |
| Diaryltriazinone | <0.05 | ND | ND | ND | ND |
| Unknown | 2.04 | <0.05 | <0.05 | <0.05 | <0.05 |
| Total Impurity | 2.21 | 0.07 | 0.12 | 0.20 | 0.21 |

Test A Conditions: 30° C. at 65% relative humidity in sealed bottles.
Test B Conditions: 40° C. at 75% relative humidity in sealed bottles.
ND: None detected.

### Example 10

The pharmaceutical composition described herein comprising a soft gelatin capsule shell and a matrix fill as described herein were prepared in a batch using the composition shown in Table 10 and the stability of dabigatran etexilate mesylate comprised by the matrix fill was tested and compared to commercially available capsules comprising dabigatran etexilate mesylate as shown in Table 14. The soft gelatin capsules described herein were stored in sealed bottles for a period of one or two months at 25° C. at 60% relative humidity (Test C).

TABLE 14
Stability and Percent Purity (%) of the Pharmaceutical
Composition Comprising Dabigatran (DEM)

| | EX Batch | Test C | Test C |
|---|---|---|---|
| Impurity | 0 Months | 1 Month | 2 Months |
| DBG-2A | <0.05 | <0.05 | <0.05 |
| DBG-3A Acid | <0.05 | <0.05 | <0.05 |
| DBG-3A Ester | <0.05 | <0.05 | <0.05 |
| DBG-3A Amide | 0.07 | 0.10 | 0.09 |
| DBG-3A Dihexyl | <0.05 | ND | <0.05 |
| DBG-3A Hexyl Amide | ND | <0.05 | <0.05 |
| DBG-3A Des Amino | ND | ND | ND |
| Pyridine | | | |
| CBA | ND | <0.05 | ND |
| DBG-3A Methoxy | ND | <0.05 | <0.05 |
| Diaryltriazinone | <0.05 | <0.05 | ND |
| Total Impurity | 0.07 | 0.10 | 0.09 |

Test C Conditions: 25° C. at 60% relative humidity in open bottles.
ND: None detected.

### Example 11

The pharmaceutical composition described herein comprising a soft gelatin capsule shell and a matrix fill as described herein were prepared in a batch using the composition shown in Table 10 and the stability of dabigatran etexilate mesylate comprised by the matrix fill was tested and compared to commercially available capsules comprising dabigatran etexilate mesylate as shown in Table 15, Table 16, and Table 17. The soft gelatin capsules described herein were stored in sealed bottles for periods between one and twelve months at varying temperature and relative humidity conditions.

TABLE 15
Stability of the Pharmaceutical Composition Comprising Dabigatran (DEM)

| | | Test Station | | | | | | | | |
|---|---|---|---|---|---|---|---|---|---|---|
| | | Stored at 40° C./75% RH | | | | Stored at 30° C./65% RH | | | | |
| | | Months | | | | | | | | |
| | Initial | 1 | 2 | 3 | 6 | 1 | 3 | 6 | 9 | 12 |
| Physical | Pass | Pass | Pass | Pass | Pass | Pass | Pass | Pass | Pass | Pass |
| Description | | | | | | | | | | |
| Physical Evaluation | Pass | Pass | Pass | Pass | Pass | Pass | Pass | Pass | Pass | Pass |
| Dabigatran | 100.1 | 97.50 | 97.10 | 96.80 | 98.70 | 0.03 | 0.03 | 0.04 | 0.04 | NP |
| Etexilate Mesylate | | | | | | | | | | |
| % Content | | | | | | | | | | |
| Water % Content | 0.03 | 0.03 | 0.03 | 0.04 | 0.04 | 105.8 | 107.7 | 106.7 | 106.2 | NP |
| BHT % Content | 103.1 | 105.8 | 100.2 | 106.2 | 109.3 | 0.03 | 0.03 | 0.04 | 0.04 | NP |

Particle Size

| | | | | | | | | | | |
|---|---|---|---|---|---|---|---|---|---|---|
| D10 | 6.09 | 7.52 | 6.75 | 13.90 | 5.88 | 6.48 | 15.54 | 9.84 | NP | NP |
| D50 | 43.65 | 47.30 | 44.09 | 52.15 | 47.91 | 42.00 | 51.58 | 50.90 | NP | NP |
| D90 | 90.48 | 93.07 | 91.46 | 96.03 | 92.85 | 86.28 | 96.93 | 93.33 | NP | NP |

Degradation Products (%)

| | | | | | | | | | | |
|---|---|---|---|---|---|---|---|---|---|---|
| DBG-2A Mesylate | <0.05 | 0.05 | 0.05 | ND | <0.05 | <0.05 | ND | <0.05 | 0.07 | 0.09 |
| DBG-3A Hexyl | ND | <0.05 | <0.05 | <0.05 | <0.05 | <0.05 | <0.05 | <0.05 | <0.05 | ND |
| Amide Mesylate | | | | | | | | | | |
| DBG-3A | ND | ND | ND | ND | ND | ND | ND | ND | ND | ND |
| Desaminopyridine | | | | | | | | | | |
| Mesylate | | | | | | | | | | |
| DBG-3A Acid | <0.05 | <0.05 | <0.05 | <0.06 | <0.05 | <0.05 | <0.05 | <0.05 | <0.05 | <0.05 |
| Mesylate | | | | | | | | | | |
| CBA Mesylate | ND | ND | ND | ND | ND | <0.05 | ND | <0.05 | ND | ND |
| DBG-3A Methoxy | ND | <0.05 | <0.05 | ND | <0.05 | ND | ND | <0.05 | <0.05 | ND |
| Mesylate | | | | | | | | | | |
| Diaryltriazinone | ND | ND | ND | <0.05 | <0.05 | ND | ND | ND | ND | ND |
| Mesylate | | | | | | | | | | |
| DBG-3A Ester | <0.05 | <0.05 | <0.05 | <0.05 | <0.05 | <0.05 | <0.05 | <0.05 | <0.05 | <0.05 |
| Mesylate | | | | | | | | | | |
| DBG-3A Amide | 0.07 | 0.15 | 0.16 | 0.15 | <0.05 | 0.12 | 0.08 | 0.14 | 0.19 | 0.25 |
| Mesylate | | | | | | | | | | |
| DBG-3A Dihexyl | <0.05 | <0.05 | <0.05 | ND | ND | ND | ND | ND | ND | ND |
| Impurity Mesylate | | | | | | | | | | |
| Largest unknown | <0.05 | <0.05 | <0.05 | ND | <0.05 | <0.05 | ND | <0.05 | — | ND |
| Impurity | | | | | | | | | | |
| Total Impurities | 0.07 | 0.20 | 0.21 | 0.21 | 0.22 | 0.12 | 0.13 | 0.07 | 0.26 | 0.34 |

Dissolution (%)

| | | | | | | | | | | |
|---|---|---|---|---|---|---|---|---|---|---|
| 10 min Apparatus-2 | 26 | 13 | 18 | 9 | | 17 | 4 | | | NP |
| 10 min Apparatus-3 | | | | 7 | 9 | | 28 | 28 | 17 | NP |
| 20 min Apparatus-2 | 66 | 46 | 53 | 30 | | 62 | 24 | | | NP |
| 20 min Apparatus-3 | | | | 42 | 62 | | 79 | 80 | 64 | NP |
| 30 min Apparatus-2 | 92 | 74 | 76 | 52 | | 89 | 58 | | | NP |
| 30 min Apparatus-3 | | | | 83 | 86 | | 87 | 92 | 87 | NP |
| 45 min Apparatus-2 | 95 | 95 | 85 | 82 | | 95 | 87 | | | NP |
| 45 min Apparatus-3 | | | | 93 | 95 | | 89 | 90 | 95 | NP |

ND: None Detected.
NP: Not Performed.

TABLE 16
Stability of the Pharmaceutical Composition Comprising
Dabigatran (DEM)

| | | Stored at 25° C./60% RH/Lot 1 | | | |
|---|---|---|---|---|---|
| | | Months | | | |
| Test Station | Initial | 3 | 6 | 9 | 12 |
| Physical Description | Pass | Pass | Pass | Pass | Pass |
| Physical Evaluation | Pass | Pass | Pass | Pass | Pass |
| Dabigatran Etexilate | 100.1 | 101.5 | 96.8 | 103.3 | 97.0 |
| Mesylate % Content | | | | | |
| Water % Content | 0.03 | 0.03 | 0.04 | 0.04 | NP |
| BHT % Content | 103.1 | 110.1 | 106.9 | 107.6 | NP |

Particle Size

| | | | | | |
|---|---|---|---|---|---|
| D10 | 6.09 | 13.78 | 7.35 | NP | NP |
| D50 | 43.65 | 51.85 | 45.83 | NP | NP |
| D90 | 90.48 | 97.38 | 89.86 | NP | NP |

Degradation Products (%)

| | | | | | |
|---|---|---|---|---|---|
| DBG-2A Mesylate | <0.05 | ND | <0.05 | 0.06 | 0.09 |
| DBG-3A Hexyl Amide | ND | <0.05 | <0.05 | <0.05 | ND |
| Mesylate | | | | | |
| DBG-3A | ND | ND | ND | ND | ND |
| Desaminopyridine | | | | | |
| Mesylate | | | | | |
| DBG-3A Acid Mesylate | <0.05 | <0.05 | <0.05 | <0.05 | <0.05 |
| CBA Mesylate | ND | ND | ND | ND | ND |
| DBG-3A Methoxy | ND | ND | <0.05 | <0.05 | ND |
| Mesylate | | | | | |
| Diaryltriazinone Mesylate | ND | <0.05 | <0.05 | ND | <0.05 |
| DBG-3A Ester Mesylate | <0.05 | <0.05 | <0.05 | <0.05 | <0.05 |
| DBG-3A Amide Mesylate | 0.07 | 0.06 | 0.11 | 0.14 | 0.19 |
| DBG-3A Dihexyl Impurity | <0.05 | ND | ND | ND | ND |
| Mesylate | | | | | |
| Largest unknown Impurity | <0.05 | ND | <0.05 | — | ND |
| Total Impurities | 0.07 | 0.11 | 0.11 | 0.20 | 0.28 |

Dissolution (%)

| | | | | | |
|---|---|---|---|---|---|
| 10 min Apparatus-2 | 26 | 8 | | 24 | NP |
| 10 min Apparatus-3 | | 27 | 10 | | NP |
| 20 min Apparatus-2 | 66 | 24 | | 87 | NP |
| 20 min Apparatus-3 | | 93 | 52 | | NP |
| 30 min Apparatus-2 | 92 | 48 | | 87 | NP |
| 30 min Apparatus-3 | | 100 | 86 | | NP |
| 45 min Apparatus-2 | 95 | 85 | | 95 | NP |
| 45 min Apparatus-3 | | 94 | 88 | | NP |

ND: None Detected.
NP: Not Performed.

TABLE 17
Stability of the Pharmaceutical Composition Comprising Dabigatran (DEM)

| | | Test Station | | | | | |
|---|---|---|---|---|---|---|---|
| | | Stored at 25° C./60% RH/Lot 2 | | | | | |
| | | Months | | | | | |
| | Initial | 1 | 2 | 3 | 6 | 9 | 12 |
| Physical Description | Pass | Pass | Pass | Pass | Pass | Pass | Pass |
| Physical Evaluation | Pass | Pass | Pass | Pass | Pass | Pass | Pass |
| Dabigatran Etexilate | 100.1 | 98.9 | 101.5 | 99.8 | 100.7 | 100.2 | 98.5 |
| Mesylate % Content | | | | | | | |
| Water % Content | 0.03 | 0.03 | 0.04 | 0.04 | 0.05 | 0.005 | NP |
| BHT % Content | 103.1 | 106.4 | 102.2 | 110.4 | 106.0 | 109.9 | NP |

Particle Size

| | | | | | | | |
|---|---|---|---|---|---|---|---|
| D10 | 6.09 | 6.958 | 6.343 | 12.525 | 6.215 | NP | NP |
| D50 | 43.65 | 46.025 | 42.625 | 50.995 | 43.897 | NP | NP |
| D90 | 90.48 | 92.136 | 87.925 | 95.646 | 87.682 | NP | NP |

Degradaton Products (%)

| | | | | | | | |
|---|---|---|---|---|---|---|---|
| DBG-2A Mesylate | <0.05 | <0.05 | <0.05 | ND | <0.05 | 0.06 | 0.08 |
| DBG-3A Hexyl Amide | ND | <0.05 | <0.05 | <0.05 | <0.05 | <0.05 | ND |
| Mesylate | | | | | | | |
| DBG-3A | ND | ND | ND | ND | ND | ND | ND |
| Desaminopyridine | | | | | | | |
| Mesylate | | | | | | | |
| DBG-3A Acid Mesylate | <0.05 | <0.05 | <0.05 | 0.06 | <0.05 | <0.05 | <0.05 |
| CBA Mesylate | ND | ND | ND | ND | ND | ND | ND |
| DBG-3A Methoxy | ND | <0.05 | <0.05 | ND | <0.05 | <0.05 | ND |
| Mesylate | | | | | | | |
| Diaryltriazinone Mesylate | ND | <0.05 | ND | <0.05 | <0.05 | ND | <0.05 |
| DBG-3A Ester Mesylate | <0.05 | <0.05 | <0.05 | <0.05 | <0.05 | <0.05 | <0.05 |
| DBG-3A Amide Mesylate | 0.07 | 0.10 | 0.09 | 0.07 | 0.12 | 0.19 | 0.26 |
| DBG-3A Dihexyl | <0.05 | ND | <0.05 | ND | ND | ND | ND |
| Impurity Mesylate | | | | | | | |
| Largest unknown Impurity | <0.05 | <0.05 | <0.05 | ND | ND | — | ND |
| Total Impurities | 0.07 | 0.10 | 0.09 | 0.13 | 0.12 | 0.25 | 0.34 |

Dissolution (%)

| | | | | | | | |
|---|---|---|---|---|---|---|---|
| 10 min Apparatus-2 | 26 | 15 | 15 | 9 | | 28 | NP |
| 10 min Apparatus-3 | | | | 37 | 70 | | NP |
| 20 min Apparatus-2 | 66 | 52 | 49 | 33 | | 89 | NP |
| 20 min Apparatus-3 | | | | 100 | 103 | | NP |
| 30 min Apparatus-2 | 92 | 88 | 82 | 66 | | 102 | NP |
| 30 min Apparatus-3 | | | | 101 | 104 | | NP |
| 45 min Apparatus-2 | 95 | 98 | 93 | 85 | | 104 | NP |
| 45 min Apparatus-3 | | | | 98 | 101 | | NP |

ND: None Detected.
NP: Not Performed.

1. An oral pharmaceutical composition comprising a soft capsule shell comprising an immediate release matrix comprising:
(a) one or more lipid or lipophilic vehicles;
(b) one or more active pharmaceutical ingredients;
(c) one or more pharmaceutically acceptable excipients; and
wherein the one or more active pharmaceutical ingredients are suspended within the matrix and the matrix inhibits moisture from contacting the active pharmaceutical ingredient.

2. The composition of claim 1, wherein the one or more active pharmaceutical ingredients comprise moisture-sensitive active ingredients, light-sensitive active ingredients, or a combination thereof.

3. The composition of claim 1, wherein the one or more active pharmaceutical ingredients comprise a moisture-sensitive active ingredient.

4. The composition of claim 3, wherein the moisture-sensitive active ingredient comprises dabigatran, aspirin, or diclofenac.

5. The composition of claim 1, wherein the active pharmaceutical ingredient comprises dabigatran.

6. The composition of claim 1, wherein the one or more lipid or lipophilic vehicles comprise about 35% to about 90% of the total mass of the matrix.

7. The composition of claim 1, wherein the one or more lipid or lipophilic vehicles comprise soybean oil, fish oil, polyunsaturated fatty acids, Miglyol® 812, Paceol™, or Capryol™, or a combination thereof.

8. The composition of claim 1, wherein the one or more active pharmaceutical ingredients comprise about 10% to about 40% of the total mass of the matrix.

9. The composition of claim 1, wherein the one or more pharmaceutically acceptable excipients comprise about 0.01% to about 15% of the total mass of the matrix.

10. The composition of claim 1, wherein the one or more pharmaceutically acceptable excipients comprises one or more non-ionic surfactants; one or more hydrophilic ionic polymers, one or more hygroscopic polymers, one or more pH buffering agents, one or more neutralizing agents, one or more antioxidants, one or more water-binding excipients, or one or more absorption enhancers, or a combination thereof.

11. The composition of claim 1, wherein the one or more pharmaceutically acceptable excipients comprises: antioxidants, non-ionic surfactants, absorption enhancers, or combinations thereof.

12. The composition of claim 11, wherein the non-ionic surfactants comprise about 5% to about 15% of the total mass of the matrix.

13. The composition of claim 11, wherein the non-ionic surfactants comprise about 35% to about 80% of the total mass of the matrix.

14. (canceled)

15. The composition of claim 11, wherein the one or more antioxidants comprises about 0.01% to about 2% of the total mass of the matrix.

16. The composition of claim 11, wherein the one or more antioxidants comprise tocopherol, BHT, BHA, citric acid, ascorbic acid, or phenolic diterpene, or a combination thereof.

17-19. (canceled)

20. The composition of claim 1, wherein the soft capsule shell comprises one or more film forming polymers, one or more plasticizers, and one or more solvents.

21. (canceled)

22. The composition of claim 1, wherein the soft capsule shell comprises:
(a) about 43% of one or more film-forming polymers;
(b) about 20% of one or more plasticizers; and
(c) about 37% of one or more solvents.

23. (canceled)

24. The composition of claim 1, wherein the matrix comprises:
(a) about 80% to about 90% soybean oil;
(b) about 9% to about 20% dabigatran; and
(c) about 0.01% BHT.

25-30. (canceled)

31. A method for manufacturing an oral immediate release soft capsule shell and matrix fill comprising:
(a) providing a matrix comprising:
(i) about 80% to about 90% soybean oil;
(ii) about 9% to about 20% dabigatran; and
(ii) about 0.01% BHT;
(b) providing a soft capsule gel mass comprising:
(i) about 43% of at least one film-forming polymer;
(ii) about 20% of at least one plasticizer; and
(iii) about 37% of a solvent;
(c) casting the soft capsule gel mass into films using heat-controlled drums or surfaces; and
(d) forming a soft capsule comprising the matrix composition using rotary die encapsulation technology.

32-35. (canceled)

36. A method for treating, ameliorating the symptoms of, or delaying the onset of a medical condition by providing a subject in need thereof with an oral pharmaceutical composition comprising:
(a) one or more lipid or lipophilic vehicles;
(b) one or more light or water sensitive active pharmaceutical ingredients; and
(c) one or more pharmaceutically acceptable excipients;
wherein the one or more active pharmaceutical ingredients are suspended within the matrix fill and the matrix inhibits moisture from contacting the active pharmaceutical ingredients.

37. The method of claim 36, wherein the medical condition comprises a coagulation disorder comprising: stroke, ischemic stroke, hemorrhagic stroke, pulmonary embolism, clotting in patients with non-valvular atrial fibrillation, stroke in patients with non-valvular atrial fibrillation, systemic embolism in patients with non-valvular atrial fibrillation, deep vein thrombosis or a combination thereof.

38-44. (canceled)
